(19) United States
(12) Patent Application Publication
(10) Pub. No.: US 2025/0289844 A1
(43) Pub. Date: Sep. 18, 2025

(54) TREATMENT OF GM1 DEFICIENCIES

(71) Applicant: Seneb BioSciences, Inc., Wilton, CT (US)

(72) Inventor: Shawn DeFrees, San Marcos, CA (US)

(21) Appl. No.: 18/861,183

(22) PCT Filed: May 1, 2023
(86) PCT No.: PCT/US2023/066437

§ 371 Date: Oct. 28, 2024

Related U.S. Application Data
(60) Provisional application No. 63/336,817, filed on Apr. 29, 2022.

(51) Int. Cl. C07H 15/10

(52) U.S. Cl. CPC C07H 15/10

(57) ABSTRACT

Highly pure, highly homogeneous ganglioside compositions are provided in addition to methods of making and using these compositions. An exemplary ganglioside composition is a GM1 composition.

### CROSS-REFERENCES TO RELATED APPLICATIONS

The present application is an International Application which claims priority to U.S. Provisional Application No. 63/336,817, filed Apr. 29, 2022, which is herein incorporated by reference in its entirety.

### FIELD OF THE INVENTION

This invention relates to a highly homogeneous synthetic GM1 composition, pharmaceutical formulations comprising this composition, methods of preparing the composition and methods of treating a disorder associated with a reduction in the glycolipid GM1 by administering an effective amount of pharmaceutical formulation of the invention.

### BACKGROUND OF THE INVENTION

Gangliosides are a class of glycolipids, often found in cell membranes, that consist of three elements. One or more sialic acid residues are attached to an oligosaccharide or carbohydrate core moiety, which in turn is attached to a hydrophobic lipid (ceramide) structure which generally is embedded in the cell membrane. The ceramide moiety includes a long chain base (LCB) portion and a fatty acid (FA) portion. Gangliosides, as well as other glycolipids and their structures in general, are discussed in, for example, Lehninger, Biochemistry (Worth Publishers, 1981) pp. 287-295 and Devlin, Textbook of Biochemistry (Wiley-Liss, 1992). Gangliosides are classified according to the number of monosaccharides in the carbohydrate moiety, as well as the number and location of sialic acid groups present in the carbohydrate moiety. In this nomenclature, G refers to ganglioside series, the second letter refers to the number of sialic acid residues (mono, di, tri, etc.), and the number (1, 2, 3, etc.) refers to the order of migration of the ganglioside on thin-layer chromatography (TLC) (e.g., GM3>GM2>GM1). Monosialogangliosides are given the designation “GM”, disialogangliosides are designated “GD”, trisialogangliosides “GT”, and tetrasialogangliosides are designated “GQ”.

Gangliosides are most abundant in the brain, particularly in nerve endings. They are believed to be present at receptor sites for neurotransmitters, including acetylcholine, and can also act as specific receptors for other biological macromolecules, including interferon, hormones, viruses, bacterial toxins, and the like. Gangliosides are have been used for treatment of nervous system disorders, including cerebral ischemic strokes. See, e g., Mahadnik et al. (1988) Drug Development Res. 15: 337-360; U.S. Pat. Nos. 4,710,490 and 4,347,244; Horowitz (1988) Adv. Exp. Med. and Biol. 174: 593-600; Karpiatz et al. (1984) Av. Exp. Med. and Biol. 174: 489-497.

Certain gangliosides are found on the surface of human hematopoietic cells (Hildebrand et al. (1972) Biochim. Biophys. Acta 260: 272-278; Macher et al. (1981) J. Biol. Chem. 256: 1968-1974; Dacremont et al. Biochim. Biophys. Acta 424: 315-322; Klock et al. (1981) Blood Cells 7: 247) which may play a role in the terminal granulocytic differentiation of these cells. Nojiri et al. (1988) J. Biol. Chem. 263: 7443-7446. These gangliosides, referred to as the “neolacto” series, have neutral core oligosaccharide structures having the formula [Galβ-(1,4)GlcNAcβ(1,3)]ₙ Galβ-(1,4)Glc, where n=1-4. Included among these neolacto series gangliosides are 3′-nLM₁ (NeuAcα (2,3)Galβ-(1,4)GlcNAcβ-(1,3)Gal.beta.(1,4)Glcβ 1,1)-Ceramide) and 6′-nLM₁ (NeuAcα(2,6)Galβ (1,4)GlcNAcβ (1,3)Gal.beta.(1,4)-Glcβ (1,1)-Ceramide).

Most ganglioside products are isolated from biological sources and are hampered by the typical drawbacks of such products, e.g., lack of purity, complex product mixtures, risk of contamination by infectious agents, complex, exhaustive isolation and purification procedures, and a complicated regulatory approval process. For example, HPLC traces of several commercially available GM1 compositions isolated from a biological source are shown in FIG. 7 (expanded in FIG. 8). As is readily apparent from the traces labeled “JLYL”, “CCXT”, and “FIDIA”, the GM1 (peak at approx. 13 min) is not insignificantly contaminated by materials eluting from the column after the GM1.

To avoid the disadvantages of biologically-derived gangliosides, chemo-enzymatic processes for the synthesis of gangliosides have been developed. FIG. 10 sets forth an HPLC trace of an exemplary chemo-enzymatically prepared GM1 preparation. GM1 is the predominant component (Peak 6), however, even this de novo preparation is significantly contaminated with other gangliosides arising from intractable impurities in the starting materials as well as side-reactions occurring during the synthesis.

Prior GM1 preparations, whether isolated from a biological source or prepared by a chemo-enzymatic synthesis method are all significantly less than 95% pure with respect to GM1 (d18:1/C18:0) content.

Ganglioside preparations are typically contaminated with from about 1% to about 5% of a ceramide analogue in which the saccharide is (Gal)ₙ-Gal-GalNAc-(Sia)-Gal-Glu-. FIG. 4 (Galₙ-GM1OS). The index n is an integer of 1 or greater. This analogue is undesirable for therapeutic administration as the potential for this structure (as opposed to Gal-GalNAc-(Sia)-Gal-Glu-; FIG. 4, GM1OS) to have unwanted or undefined biological effects. Furthermore, formulations having multiple forms of the complex saccharides face a complicated regulatory pathway.

The use of gangliosides as therapeutic reagents, as well as the study of ganglioside function, would be facilitated by convenient and efficient methods of synthesizing highly pure gangliosides of a substantially uniform structure. A combined enzymatic and chemical approach to synthesis of 3′-nLM₁ and 6′-nLM₁ has been described (Gaudino and Paulson (1994) J. Am. Chem. Soc. 116: 1149-1150). However, this and other previously available synthetic methods for ganglioside synthesis suffer from low efficiency and other drawbacks. Thus, a need exists for more efficient methods for synthesizing gangliosides. The present invention fulfills this and other needs.

### SUMMARY OF THE INVENTION

The present invention provides highly pure ganglioside compositions, methods of using the gangliosides in these compositions in therapeutic modalities and methods of making these compositions. Also provided are concentrated aqueous pharmaceutical formulations of the highly pure gangliosides.

In one embodiment, the invention provides a ganglioside composition in which the sphingosine moiety of the ganglioside is present in a purity of at least 96%, at least about 97%, at least about 98% or at least about 99%. The compositions of the invention include a ganglioside fraction in which more than about 96% of the sphingosine moiety present is in a single form, i.e., the composition comprises a ganglioside fraction in which a single form of sphingosine is present in at least about 96% abundance, at least about 97% abundance, at least about 98% abundance or at least about 99% abundance. In an exemplary embodiment, the single form is a single stereoisomer. In an exemplary embodiment, the ganglioside is GM1.

In an exemplary embodiment, the content of each contaminant in the ganglioside composition of the invention is not more than about 0.2%. In a more preferred embodiment, the content each contaminant in the GM1 composition is not more than about 0.1%.

In an exemplary embodiment, the invention provides a ganglioside composition having a ganglioside fraction in which sphingosine is present in the ganglioside as a substantially pure stereoisomer, the composition having at least 98%, at least about 99% of a single stereoisomer. In an exemplary embodiment, the ganglioside is GM1.

In an exemplary embodiment, the content of each contaminant in the GM1 ganglioside composition of the invention is not more than about 0.2%. In a more preferred embodiment, the content each contaminant in the GM1 composition is not more than about 0.1%.

In an exemplary embodiment, the invention provides a ganglioside composition in which a substantial fraction of the saccharide population does not include a (Gal)ₙ-moiety attached to the GalNAc of the ceramide Gal-GalNAc-(Sia)-Gal-Glu saccharide. The index n is an integer of 1 or greater. Thus, the invention provides a ganglioside composition in which the improvement is the absence of a (Gal)ₙ moiety from the terminal Gal of the ceramidyl saccharide Gal-GalNAc-(Sia)-Gal-Glu. In various embodiments, the (Gal)ₙ moiety is absent from at least about 99% of the ganglioside, at least about 98%, at least about 96%, at least about 92%, at least about 90%, at least about 85% or at least about 80% of the ganglioside.

In an exemplary embodiment, the ganglioside composition from which the (Gal) n moiety is substantially absent from the terminal Gal of the ceramidyl saccharide Gal-GalNAc-(Sia)-Gal-Glu is a GM1 preparation.

In various embodiments, the ganglioside composition is GM1. The sphingosine d18:1 content in the GM1 of the composition is at least about 96%, at least about 97%, at least about 98%, or at least about 99% of the total weight of the GM1 in the composition.

In various embodiments, the ganglioside composition is GM1. The sphingosine d20:1 content in the GM1 of the composition is at least about 96%, at least about 97%, at least about 98%, or at least about 99% of the total weight of sphingosine in the GM1 in the composition.

In various embodiments, the ganglioside composition is GM1. The sphingosine is selected from d14:1, d15:1, d16:1, d17:1, d19:1, d21:1, d22:1 and a combination thereof in the GM1 of the composition is at least about 96%, at least about 97%, at least about 98%, or at least about 99% of the total weight of the sphingosine in the GM1 in the composition.

In various embodiments, the ganglioside composition is GM1, and at least about 96%, at least about 97%, at least about 98%, or at least about 99% of the total weight of sphingosine in the GM1 in the composition is D-erythro-sphingosine. In an exemplary embodiment, the sphingosine is d18:1.

In various embodiments, the ganglioside composition is GM1, and at least about 96% of the sphingosine is D-ethryo-, and further, a member selected from;
no more than about 0.5% of D-threo-;
no more than about 0.5% L-threo-;
no more than about 0.5% L-erythro-; and a combination thereof.

In various embodiments, the ganglioside composition is GM1. The fatty acid content in the GM1 of the composition is at least about 96%, at least about 97%, at least about 98%, or at least about 99% of the total weight of the GM1 in the composition selected from fatty acid (C12:0), (C13:0), (C14:0), (C15:0), (C16:0), (C17:0), (C18:0), (C19:0), (C20:0), (C21:0), (C22:0), and a combination thereof. In various embodiments, the fatty acid in the GM1 of the composition is at least about 96%, at least about 97%, at least about 98%, or at least 99% of the total weight of the GM1 in the composition wherein the fatty acid is a hydrogen (H) thereby forming lyso-GM1.

In various embodiments, the ganglioside composition is GM1. The unsaturated fatty acid content in the GM1 of the composition is at least about 96%, at least about 97%, at least about 98%, or at least about 99% of the total weight of the GM1 in the composition selected from fatty acid (C12:1), (C13:1), (C14:1), (C15:1), (C16:1), (C17:1), (C18:1), (C19:1), (C20:1), (C21:1) and (C22:1). In a preferred embodiment, the unsaturated fatty acid is selected from oleic acid, elaidic acid, vaccenic acid, palmitoleic acid, myristoleic acid, gadoleic acid, eicosenoic acid. In a more preferred embodiment, the unsaturated fatty acid is oleic acid.

In various embodiments, the ganglioside composition is GM1. The fatty acid and unsaturated fatty acid content of the GM1 composition contains no more than about 3.0% of a hydrocarbon chain length that is two carbons more and/or no more than about 3.0% of a hydrocarbon chain length that is two carbons less of the fatty acid chain length attached to GM1. The fatty acid and unsaturated fatty acid content of the GM1 composition contains no more than about 1.0% of a hydrocarbon chain length that is one carbon more and/or no more than about 1.0% of a hydrocarbon chain length that is one carbon less of the fatty acid chain length attached to GM1. In a preferred embodiment, the fatty acid of the GM1 composition contains a hydrocarbon chain length that is no more than about 0.5% of two carbons more and/or two carbons less of the fatty acid chain length attached to GM1.

An exemplary embodiment of the GM1 composition of the invention comprises a GM1 fraction having the following characteristics:
Sphingosine (d18:1)—at least about 96%; and a member selected from:
   Sphingosine (d17:1)—no more than about 0.1%;
   Sphinganine (d18:0)—no more than about 2%;
   Sphinganine (d20:0 —NH₂)—no more than about 0.5%;
   Sphingosine (d22:1 —NH₂)—no more than about 0.5%,
      and a combination thereof;
Stearic acid (C18:0)—at least about 92%; and a member selected from:
   Palmitic acid (C16:0)—no more than about 3%;
   Margaric acid (C17:0)—no more than about 1%;
   Nonadecylic acid (C19:0)—no more than about 0.2%;
   Arachidic acid (C20:0)—no more than about 2%
      and a combination thereof.

An exemplary embodiment of the GM1 composition of the invention comprises a GM1 fraction having the following characteristics:
Sphingosine (d20:1)—at least about 96%; and a member selected from:
   Sphinganine (d20:0)—no more than about 2.5%;
   Sphingosine (d18:1)—no more than about 0.4%;
   Sphingosine (d16:1)—no more than about 0.4%;
   Sphingosine (d22:1)—no more than about 2%;
      and a combination thereof;
Stearic acid (C18:0)—at least about 92%; and a member selected from:
   Palmitic acid (C16:0)—no more than about 3%;
   Margaric acid (C17:0)—no more than about 1%;
   Nonadeclic acid (C19:0)—no more than about 0.2%;
   Arachidic acid (C20:0)—no more than about 2%
      and a combination thereof.

An exemplary embodiment of the GM1 composition of the invention comprises a GM1 fraction having the following characteristics:
Sphingosine (d19:1)—at least about 96%; and a member selected from:
   Sphinganine (d19:0)—no more than about 2.5%;
   Sphingosine (d18:1)—no more than about 0.4%;
   Sphingosine (d17:1)—no more than about 0.4%;
   Sphinganine (d18:0)—no more than about 2%;
   Sphinganine (d20:0)—no more than about 0.5%;
   Sphingosine (d20:1)—no more than about 0.5%,
      and a combination thereof;
Stearic acid (C18:0)—at least about 92%; and a member selected from:
   Palmitic acid (C16:0)—no more than about 3%;
   Margaric acid (C17:0)—no more than about 1%;
   Nonadecylic acid (C19:0)—no more than about 0.2%;
   arachidic acid (C20:0)—no more than about 2%
      and a combination thereof.

An exemplary embodiment of the GM1 composition of the invention comprises a GM1 fraction having the following characteristics:
Sphingosine (d17:1)—at least about 96%; and a member selected from:
   Sphinganine (d17:0)—no more than about 2.5%;
   Sphingosine (d18:1)—no more than about 0.4%;
   Sphingosine (d16:1)—no more than about 0.4%;
   Sphinganine (d18:0)—no more than about 2%;
   Sphinganine (d19:0)—no more than about 0.5%;
      and a combination thereof;
Stearic acid (C18:0)—at least about 92%; and a member selected from:
   Palmitic acid (C16:0)—no more than about 3%;
   Margaric acid (C17:0)—no more than about 1%;
   Nonadecylic acid (C19:0)—no more than about 0.2%;
   Arachidic acid (C20:0)—no more than about 2%
      and a combination thereof.

An exemplary embodiment of the GM1 composition of the invention comprises a GM1 fraction having the following characteristics:
Sphingosine (d19:1)—at least about 96%; and a member selected from:
   Sphinganine (d19:0)—no more than about 2.5%;
   Sphingosine (d18:1)—no more than about 0.4%;
   Sphingosine (d20:1)—no more than about 0.4%;
   Sphinganine (d18:0)—no more than about 2%;
   Sphinganine (d20:0)—no more than about 0.5%;
      and a combination thereof;
Stearic acid (C18:0)—at least about 92%; and a member selected from:
   Palmitic acid (C16:0)—no more than about 3%;
   Margaric acid (C17:0)—no more than about 1%;
   Nonadecylic acid (C19:0)—no more than about 0.2%;
   Arachidic acid (C20:0)—no more than about 2%
      and a combination thereof.

An exemplary embodiment of the GM1 composition of the invention comprises a GM1 fraction having the following characteristics:
Sphingosine (d18:1)—at least about 96%; and a member selected from:
   Sphingosine (d17:1)—no more than about 0.1%;
   Sphinganine (d18:0)—no more than about 2%;
   Sphinganine (d20:0 —NH₂)—no more than about 0.5%;
   Sphingosine (d22:1 —NH₂)—no more than about 0.5%,
      and a combination thereof;
Stearic acid (C18:0)—at least about 92%; and a member selected from:
   Palmitic acid (C16:0)—no more than about 3%;
   Margaric acid (C17:0)—no more than about 1%;
   Nonadecyclic acid (C19:0)—no more than about 0.2%;
   Arachidic acid (C20:0)—no more than about 2%
      and a combination thereof;
Sphingosine (d18:1) chirality—at least about 96% D-erythro- and a member selected from:
   Sphingosine (d18:1) chirality—no more than about 0.5% of D-threo-;
   Sphingosine (d18:1) chirality—no more than about 0.5% L-threo-;
   Sphingosine (d18:1) chirality—no more than about 0.5% L-erythro-;
      and a combination thereof.

An exemplary embodiment of the GM1 composition of the invention comprises a fraction having the following characteristics:
Sphingosine (d20:1)—at least about 96%; and a member selected from:
   Sphinganine (d20:0)—no more than about 2.5%;
   Sphingosine (d18:1)—no more than about 0.4%;
   Sphingosine (d16:1)—no more than about 0.4%;
   Sphingosine (d22:1)—no more than about 2%;
      and a combination thereof;
Stearic acid (C18:0)—at least about 92%; and a member selected from:
   Palmitic (C16:0)—no more than about 3%;
   Margaric acid (C17:0)—no more than about 1%;
   Nonadecylic acid (C19:0)—no more than about 0.2%;
   Arachidic acid (C20:0)—no more than about 2%
      and a combination thereof;
Sphingosine (d20:1) chirality—at least about 96% D-erythro- and a member selected from:
   Sphingosine (d20:1) chirality—no more than about 0.5% of D-threo-;
   Sphingosine (d20:1) chirality—no more than about 0.5% L-threo-;
   Sphingosine (d20:1) chirality—no more than about 0.5% L-erythro-;
      and a combination thereof.

In an exemplary embodiment, the GM1 fraction described above is at least about 80%, at least about 85%, at least about 90%, at least about 92%, at least about 94%, at least about 96%, at least about 98%, or at least about 99% of the total weight of the GM1 in the composition.

In various embodiments, the ganglioside composition of the invention is in the form of a pharmaceutical formulation and the ganglioside composition is combined with one or more diluent, carrier, additive, excipient, etc. Design of appropriate formulations is within the abilities of those of ordinary skill in the art. In an exemplary embodiment, the ganglioside is GM1, e.g., a GM1 described above.

In an exemplary embodiment, the ganglioside is in the form of a clear, colorless aqueous pharmaceutical formulation comprising the ganglioside substantially entirely dissolved in water, the formulation optionally comprising one or more additives, or excipient, wherein the formulation is devoid of organic solvents and cosolvents, the formulation comprising from about 50 mg/mL to about 500 mg/mL of the ganglioside (e.g., GM1).

In other exemplary embodiments, the ganglioside is in an aqueous pharmaceutical formation comprising the ganglioside entirely dissolved in water, the formulation optionally comprising one or more additives, excipient, or cosolvents, the formulation comprising from about 50 mg/mL to about 500 mg/mL of the ganglioside (e.g., GM1).

In various embodiments, the invention provides a surprising advance in ganglioside formulations. Gangliosides isolated from natural sources, e.g., GM1, are recognized in the literature as being only sparingly soluble in water, e.g., not more than about 50 mg/mL, unless formulated as a liposome with other more water-soluble components. For example, one preparation of GM1 is disclosed as being soluble to only 3 mg/ml water (Cayman Chemical Product Information, Item No. 19579). Another GM1 preparation from bovine brain is disclosed as only “slightly soluble in water (micellar aggregates)”, (USBiological Life Sciences, G2006-10; CAS: 37758-47-7). Other references describe GM1 as having effectively no solubility in water ((http://www.metabolomicscentre.ca?utm_source=hmdb&utm_medium-banner&utm_campaign=tmic-campaign)).

In contrast to the findings of the literature, exemplary highly pure, structurally homogeneous gangliosides of the invention are readily formulated into highly concentrated, clear, non-cloudy, colorless aqueous solutions. In various embodiments, the concentration of the aqueous formulation of GM1 of the invention is at least about 50 mg/mL. Exemplary formulation concentrations include from about 50 to about 100 mg/mL, about 100 to about 150 mg/mL, about 150 to about 200 mg/mL, about 200 to about 250 mg/mL, about 250 to about 300 mg/mL, about 300 to about 350 mg/mL, about 350 to about 400 mg/mL and about 400 to about 500 mg/mL in GM1. In an exemplary embodiment, the aqueous formulation of GM1 is about 100 mg/mL, about 150 mg/mL, about 200 mg/mL, about 250 mg/mL, or about 300 mg/mL in GM1. In a still more preferred embodiment, the aqueous formulations of GM1 are administered parenterally (i.e., subcutaneous, intraperitoneal, intravenous).

In an exemplary embodiment, the ganglioside solution includes no organic solvent or cosolvent, and the solvent is solely water. In various embodiments, the solutions of the gangliosides show little to no light absorption at 600 nm, indicative of the absence of particles, e.g., micelles, aggregates, precipitates, which are known to form with gangliosides in water. In an exemplary embodiment, not more than about 20%, e.g., 10%, e.g., 5% of light at 600 nm incident on the solution of the invention is absorbed. In various embodiments not more than about 2%, 4%, 6%, 8% or 10% of light at 600 nm incident on the solution of the invention is absorbed. The optical density at 600 nm is a good measure of how turbid or how much scattering material (such as ganglioside micelles, aggregates, precipitates) are in a solution, generally, the more precipitates the higher the absorbance. This is readily determined, e.g., using an available UV/Vis spectrophotometer.

FIG. 26. summarizes exemplary solubility of the GM1 of the invention at various concentrations of GM1, and plasma concentration of the GM1 in Sprague Dawley after subcutaneous administration at the provided concentrations. Formulations of the GM1 of the invention were prepared including GM1 from about 50 mg/mL to about 500 mg/mL in water or buffer with or without excipients, or organic solvents and/or cosolvents.

In an exemplary embodiment, the ganglioside formulation of the invention is formulated for parenteral administration.

In various embodiments, the ganglioside in the ganglioside preparation of the invention penetrates the blood brain barrier of a mammal. In various embodiments, the ganglioside preparation penetrates the blood brain barrier in the experiment described in FIG. 16. (Wu (2011) Neurochem. Res. 36(9):1706-1714). In an exemplary embodiment, the ganglioside preparation is a preparation of GM1, e.g., a GM1 preparation of the invention.

In an exemplary embodiment, the invention provides a GM1 composition, which, when assayed by HPLC according to the conditions set out in Example 3, provides the chromatogram shown in FIG. 7 and expansion FIG. 8B.

In various embodiments, the invention provides a GM1 composition, which, when assayed by HPLC according to the conditions set forth in Example 3, provides the chromatogram according to FIG. 10 in which one or more of peaks 5, 8, 10, 11 or 12, in any combination, are substantially absent from the chromatogram. In various embodiments, the invention provides a GM1 composition, which, when assayed by HPLC according to the conditions set forth in Example 3, provides the chromatogram according to FIG. 10 in which each of peaks 5, 8, 10, 11 or 12, in any combination, are substantially absent from the chromatogram. In various embodiments, the chromatogram is displayed at a level of magnification at which peak 6 is designated as 100% intensity (relative abundance).

Exemplary methods of preparing the ganglioside compositions of the invention involve the enzymatic transfer of carbohydrates, including sialic acids, to a highly pure, highly homogeneous sphingosine precursor. In particular, the methods involve contacting the sphingosine precursor with one or more glycosyltransferases, the corresponding sugar donor(s) for the glycosyltransferases, and other reactants required for transfer of a selected glycosyl moiety via the glycosyltransferase activity, for a sufficient time and under appropriate conditions to transfer the sugar or sugars from the donor moiety to the sphingosine precursor. In some embodiments, one or more of the enzymatic reactions is carried out in the presence of an organic solvent, which increases the efficiency of the glycosylation reaction.

In an exemplary embodiment, the sphingosine is assembled from highly pure precursors, each of which is essentially a single compound, e.g., single isomer, e.g., single stereoisomer. FIG. 2 and FIG. 3. Exemplary precursors have up to about 6% stereoisomeric or other impurity, up to about 5%, up to about 4%, up to about 3%, up to about 2% or up to about 1% of such impurity(ies).

In an exemplary embodiment, the method of the invention includes the improvement of contacting a ganglioside preparation with a galactosidase, thereby removing (Gal)ₙ from the terminal Gal of the ceramidyl saccharide. In an exemplary embodiment, the ganglioside is a GM1 preparation, e.g., a GM1 preparation of the invention, and it is contacted with a galactosidase, thereby converting Galₙ-GM1OS to GM1OS. Exemplary galactosidases of use in this embodiment include those set forth in FIG. 17. This embodiment of the invention can also be performed on (Gal)-GM1, itself.

The invention will be better understood from review of the Detailed Description following.

### BRIEF DESCRIPTION OF THE DRAWINGS

FIG. 1. Synthesis of D-erythro-sphingosine.

FIG. 2A. Chiral purity of D-erythro-sphingosine after recrystallization. Purity was determined using an HPLC-MS/MS method using a Regis Chirosil RCA, 4.6×250 mm, 5 μm column with acetic acid-acetonitrile elution monitored at 200 nm. FIGS. 2B and 2C. Achiral purity of D-erythro-sphingosine after recrystallization. Purity was determined using an HPLC-MS method using an Agilent Poroshell 120 PFP, 3.0×100 mm, 2.7 μm column with TFA-acetonitrile elution monitored at 200 nm.

FIG. 3. Purity of stearic anhydride before and after recrystallization. Purity was determined using an HPLC-MS method. The HPLC method used a C18 silica column with dichloromethane/isopropanol (30/70) elution monitored at 200 nm. Lot 1 is crude stearic anhydride and Lot 2 is recrystallized. Activated stearic acid can be used as the starting material for the recrystallization.

FIG. 4. Manufacture of GM1 oligosaccharide (GM1OS). An engineered E. coli was used to prepare GM1 oligosaccharide. The crude GM1 oligosaccharide was treated with galactosidase to remove the extra galactose from the hypergalactosylated-GM1 oligosaccharide (Galn-GM1OS; n=1-3) to create GM1OS.

FIG. 5. Manufacture of GM1 oligosaccharide α-fluoride (GM1OS-F). GM1OS is converted to GM1OS-F using a three-step process including acetylation, fluorination and deacetylation.

FIG. 6. Manufacture of GM1 (d18:1/C18:0). GM1OS-F is converted to lyso-GM1 by addition of D-erythro-sphingosine (d18:1) using a mutant endoglycoceramidase (mEGCase) and addition of stearic acid (C18:0). Alternatively, GM1 (d20:1/C18:0) is prepared by addition of D-erythro-sphingosine (d20:1) to GM1OS-F using a mutant endoglycoceramidase and addition of stearic acid (C18:0).

FIG. 7. HPLC chromatogram comparing GM1 from various sources. Jilin Yinglian Biopharma (JLYL), Changchung Xianglong Pharma (CCXT), Fidia (Sygen), GM1 (d18:1/C18:0).

FIG. 8. HPLC chromatogram (enlarge between 10 to 20 minutes) comparing GM1 from various sources. Jilin Yinglian Biopharma (JLYL), Changchung Xianglong Pharma (CCXT), Fidia (Sygen), GM1 (d18:1/C18:0).

FIG. 9. Comparison of chromatograms and characterization of peaks from different GM1 sources using HPLC-MS/MS. Jilin Yinglian Biopharma (JLYL), Changchung Xianglong Pharma (CCXT), Fidia (Sygen), GM1 (d18:1/C18:0). Grey box indicates no peak detected.

FIG. 10. HPLC Chromatogram of GM1 prepared by an earlier chemo-enzymatic process, FIGS. 4-6. The sphingosine and stearic acid used in the process were commercially available materials that contained mixtures of lipid chain length and stereoisomers. The GM1 was purified at scale using reversed phase chromatography (small bead size and step elution) and the eluted product peak fractionated to provide GM1. Peak identification was performed using HPLC-MS/MS analysis.

FIG. 11. Different GM1 species, lipid heterogeneity and glycan heterogeneity, are formed using the synthetic process when the starting reagents are not a single species. Difficulties in separating the different ganglioside including lipid and glycan variants is demonstrated by the inability to purify naturally derived gangliosides such as GM1 for therapeutic uses, FIGS. 7-10. Coupling of GM1OSF and (Gal)ₙ-GM1OSF to sphingosine using EGCase as in FIG. 6 produced GM1 (d18:1/C18:0) and (Gal)ₙ-GM1 (d18:1/C18:0) which were not completely separated using any chromatography method. Coupling of GM1OS to sphingosine (a mixture of different chain lengths) using EGCase followed by stearic acid addition produced GM1 (d18:1/C18:0) as the major product and GM1 (tetra18:1/C18:0), GM1 (tetra18:0/C18:0), GM1 (d17:1/C18:0) and GM1 (d16:0/C18:0) along with many other glycoforms after purification, FIG. 10. Coupling of GM1OS to sphingosine using EGCase followed by stearic acid addition (90% stearic acid C18:0) produced GM1 (d18:1/C18:0) as the major product and GM1 (d18:1/C20:0), and GM1 (d18:1/C17:0) after purification, FIG. 10. The purification of GM1 species is complicated further when the sphingosine is not a single enantiomeric species prior to coupling with GM1OSF.

FIG. 12. Comparison of GM1 prepared by the earlier process GM1 and GM1 (d18:1/C18:0) prepared using the new process. FIG. 12A. GM1 produced by the original process. FIG. 12B. GM1 (d18:1/C18:0) produced by the improved process.

FIG. 13. Single GM1 species manufactured using the synthetic process when the starting reagents are a single species.

FIG. 14. Motor and Cognitive function of GM2 synthase deficient mice (heterozygotes; HT and homozygotes; KO; 200-300 day old) treated with either saline (Sal) or GM1 (d18:1, C18:0) (30 mg/Kg, intraperitoneal, once daily) and compared to age matched controlled wild type mice (WT). FIG. 14A. Mouse grip strength, and FIG. 14B. Mouse cognitive function (T-maze), were assessed after 3 weeks of dosing.

FIG. 15. Motor and Cognitive function of GM3 synthase deficient mice (heterozygotes; HT and homozygotes; KO; 7-10 months old) treated with either saline (Sal) or GM1 (d18:1, C18:0) (30 mg/Kg, intraperitoneal, once daily for 3 weeks) and compared to age matched controlled wild type mice (WT). FIG. 15A. Grip Duration, FIG. 15B. Beam Transversal, and FIG. 15C. Short-term memory impairment (T-maze).

FIG. 16. Brain penetration of GM1 (d18:1/C18:0) in GM2 synthase deficient mice (homozygotes; KO), 200-300 day old, treated GM1 (30 mg/Kg, intraperitoneal, once daily) and compared to age matched controlled wild type mice (WT). FIG. 16A. Histological analysis of the presence of GM1 in the substantia nigra. GM1 was visualized using fluorescein labeled cholera toxin b. The presence of GM1 stains green. The box indicates the substantia nigra pars compacta region. FIG. 16B. Quantitative TLC (silica) analysis of GM1 and other gangliosides in the mouse cortex at day 5. Mice were treated with either GM1 (d18:1/C18:0) or saline (Sal). Standards are STD.

FIG. 17. The β-gluco/galactosidases capable of removing the excess galactose residue from (Gal)ₙ-GM1OS and other intermediates used in the GM1 (d18:1/C18:0) manufacturing process.

FIG. 18. Ganglioside biosynthetic pathway. The first step in producing the a-, b- and c-series gangliosides is glycosylation of lactosylcermide (LacCer) with ST3Gal5. Supplemented gangliosides to cells can be glycosylated by the ganglioside biosynthetic pathways in the endoplasmic reticulum and golgi to produce higher ganglioside glycoforms that include a-, b-and c-series gangliosides, e.g., GM1 can be converted to GD1a or GT1b and GM3 can be converted to GM2, GD3 and GM1.

FIG. 19. Ganglioside degradation and salvage pathways. Gangliosides are metabolized by enzyme of the ganglioside degradation pathway to lower glycoforms in the endosomal/lysosomal system, e.g., GM1 is metabolized to GM2 and GM3. The lower ganglioside glycoforms can be shuttled intracellularly to the endoplasmic reticulum and/or golgi where the gangliosides can be reglycosylated using glycosyltransferases of the ganglioside biosynthetic pathway, FIG. 18, to produce the a-, b- or c-series gangliosides, e.g., GM3 can be glycosylated to produce higher ganglioside glycoforms such as GD3, GD2, GM2 and GM1. This process is the salvage pathway.

FIG. 20. Diseases associated with a ganglioside deficiency, e.g. GM1, caused by a reduction in GM3, GM2 and/or GM1 synthase activity.

FIG. 21. GM1 levels in the substantia nigra pars compacta of GM2 synthase knockout heterozygote (HT) mice and wild type (WT) mice. (♦) wild type mice. (□) GM2 synthase knockout (HT) mice.

FIG. 22. GM1 levels of dopaminergic neurons (TH+; tyrosine hydroxylase positive) in the substantia nigra pars compacta from human idiopathic Parkinson's disease brain. DA is dopaminergic; aSyn is alpha-synuclein; (▪) is the substantia nigra pars compacta region of healthy human age-matched brains; (□) is the substantia nigra pars compacta region of idiopathic Parkinson's disease brain.

FIG. 23. Clinical Studies of Parkinson's disease using brain derived GM1. The first study was an open-label study administering brain derived GM1 (1,000 mg; IV) once and then (200 mg/day, SC) for 5 years monitoring the subjects UPDRS motor scores (□); hashed line. The second study was a delayed-start study administering brain derived GM1 (200 mg/day, SC) according to study protocol and study arm. Response was measured using UPDRS motor scores; placebo arm (▴) delayed-start arm (●) and early-start arm (▪), solid lines.

FIG. 24. Gene expression and GM1 levels in the human caudate of Huntington's disease subjects. FIG. 24A. Gene Expression in Huntington's disease caudate. FIG. 24B. Relative amount of GM1 level reduction in Huntington's disease mice and Huntington's disease human caudate.

FIG. 25. Improvement of motor function in Huntington's disease YAC128 mice after ganglioside replacement therapy using ganglioside compositions of the invention. Mice were administered GM1 using an Alzet pump and intracerebral ventricular injection. FIG. 25A. Improved rotarod motor function in YAC128 mice after ganglioside replacement therapy; WT mice treated with GM1 (□), solid line; WT mice treated with cerebral spinal fluid (▪) hashed line; YAC128 Huntington's disease mice treated with cerebral spinal fluid, (⋄), hashed line; YAC128 Huntington's disease mice treated with GM1 (♦), solid line. FIG. 25B. Improved narrow beam motor function in YAC128 mice after ganglioside replacement therapy. WT mice treated with GM1 (□), solid line; WT mice treated with cerebral spinal fluid (▪) hashed line; YAC128 Huntington's disease mice treated with cerebral spinal fluid (⋄) hashed line; YAC128 Huntington's disease mice treated with GM1 (♦) solid line.

FIG. 26. Solubility of GM1 of the Invention and Pharmacokinetics of different formulations of GM1 in Sprague Dawley after subcutaneous administration at a dose of 30 mg/Kg. GM1 was formulated at different GM1 concentrations from 50 mg/mL to 500 mg/mL in water or buffer with or without excipients and cosolvents.

FIG. 27. Minimum effective dose. Motor function of GM2 synthase deficient mice (heterozygotes; HT, 200-300 day old) treated with GM1(d18:1, C18:0) (intraperitoneal, once daily). FIG. 27A. Mouse grip strength, FIG. 27B. Adhesive removal, and FIG. 27C. Pole climbing.

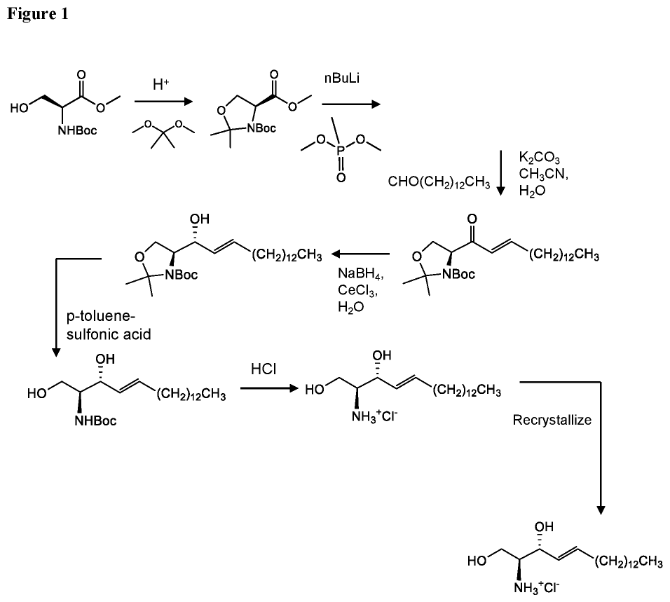

### DETAILED DESCRIPTION

### Introduction

The present invention provides highly pure ganglioside compositions, methods of using the gangliosides in these compositions in therapeutic modalities and methods of making these compositions. The compositions of the invention are also substantially homogenous with respect to the structure of the sphingoid (e.g., ceramide) and the saccharyl moiety to which the sphingoid is attached. Prior methods of isolating gangliosides from natural products, and chemo-enzymatic methods of synthesizing these compounds are unable to provide gangliosides with the levels of purity and homogeneity provided by the instant compositions and methods of making these compositions.

### Definitions

The articles “a” and “an” are used herein to refer to one or to more than one (i.e. to at least one) of the grammatical object of the article. By way of example, “an element” means one element or more than one element.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, and components have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For instance, a first property could be termed a second property, and, similarly, a second property could be termed a first property, without departing from the scope of the present disclosure. The first property and the second property are both properties, but they are not the same property.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The foregoing description included example systems, methods, techniques, instruction sequences, and computing machine program products that embody illustrative implementations. For purposes of explanation, numerous specific details are set forth in order to provide an understanding of various implementations of the inventive subject matter. It will be evident, however, to those skilled in the art that implementations of the inventive subject matter may be practiced without these specific details. In general, well-known instruction instances, protocols, structures, and techniques have not been shown in detail.

The foregoing description, for purpose of explanation, has been described with reference to specific implementations. However, the illustrative discussions below are not intended to be exhaustive or to limit the implementations to the precise forms disclosed. Many modifications and variations are possible in view of the above teachings. The implementations are chosen and described in order to best explain the principles and their practical applications, to thereby enable others skilled in the art to best utilize the implementations and various implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case-and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. Where particular values are described in the application and claims, unless otherwise stated, the term “about” means within an acceptable error range for the particular value. The term “about” can have the meaning as commonly understood by one of ordinary skill in the art. The term “about” can refer to ±10%. The term “about” can refer to ±5%.

The following abbreviations are used herein: Gal=galactosyl; GalNAc=N-acetylgalactosaminyl; Glc-glucosyl; GlcNAc=N-acetylglucosaminyl; and NeuAc, Sia =sialyl (typically N-acetylneuraminyl).

Oligosaccharides are considered to have a reducing end and a non-reducing end, whether or not the saccharide at the reducing end is in fact a reducing sugar. In accordance with accepted nomenclature, oligosaccharides are depicted herein with the non-reducing end on the left and the reducing end on the right. All oligosaccharides described herein are described with the name or abbreviation for the non-reducing saccharide (e.g., Gal), followed by the configuration of the glycosidic bond (α or β), the ring bond, the ring position of the reducing saccharide involved in the bond, and then the name or abbreviation of the reducing saccharide (e.g., GlcNAc). The linkage between two sugars may be expressed, for example, as 2,3, 2>3, 2-3, or (2,3). Each saccharide is a pyranose.

A “sphingoid,” as used herein, includes sphingosines, phytosphingosines, sphinganines, ceramides, and the like. Both naturally occurring and synthetically produced compounds are included.

A “glycosphingolipid” is a carbohydrate-containing derivative of a sphingoid or ceramide. The carbohydrate residue is attached by a glycosidic linkage to O-1 of the sphingoid.

The term “sialic acid” (abbreviated “Sia”) refers to any member of a family of nine-carbon carboxylated sugars. The most common member of the sialic acid family is N-acetyl-neuraminic acid (2-keto-5-acetamindo-3,5-dideoxy-D-glycero-D-galactononulopyranos-1-onic acid (often abbreviated as Neu5Ac, NeuAc, or NANA). A second member of the family is N-glycolyl-neuraminic acid (Neu5Gc or NeuGc), in which the N-acetyl group of NeuAc is hydroxylated. A third sialic acid family member is 2-keto-3-deoxy-nonulosonic acid (KDN) (Nadano et al. (1986) J. Biol. Chem. 261: 11550-11557; Kanamori et al. (1990) J. Biol. Chem. 265: 21811-21819. Also included are 9-substituted sialic acids such as a 9-O—C₁-C₆ acyl-Neu5Ac like 9-O-lactyl-Neu5Ac or 9-O-acetyl-Neu5Ac, 9-deoxy-9-fluoro-Neu5Ac and 9-azido-9-deoxy-Neu5Ac. For review of the sialic acid family, see, e.g., Varki (1992) Glycobiology 2: 25-40; Sialic Acids: Chemistry, Metabolism and Function, R. Schauer, Ed. (Springer-Verlag, New York (1992). The synthesis and use of sialic acid compounds in a sialylation procedure is described in, for example, international application WO 92/16640, published Oct. 1, 1992.

The term “isolated” is meant to refer to material which is substantially or essentially free from components which normally accompany the material as found in its native state. Thus, in some embodiments, the gangliosides and other glycosphingoids made using the methods of the invention do not include materials normally associated with the in situ environment (e.g., reaction medium) of these compounds. “Isolated” and “pure” are used interchangeably herein. Typically, isolated glycoconjugates of the invention are at least about 80% pure, usually at least about 90%, and preferably at least about 95% pure as measured by band intensity on a silver stained gel or other method for determining purity. Purity or homogeneity can be indicated by a number of means well known in the art, such as are described below.

“Substantially”, as used herein refers to at least about 80%, at least about 85%, at least about 90%, at least about 92%, at least about 94%, at least about 96%, at least about 98%, or at least about 90% of the total weight of the ganglioside in the composition.

“Homogeneity,” refers to the structural consistency across a population of gangliosides in a composition of the invention. Thus, a ganglioside composition of the invention in which each sphingosine moiety across members of the population of gangliosides is the same and each sugar moiety conjugated to the sphingosine moiety across members of the population is the same is said to be 100% homogenous. Homogeneity is typically expressed as a range. The lower end of the range of homogeneity for the gangliosides is about 60%, about 70% or about 80% and the upper end of the range of homogeneity is about 70%, about 80%, about 90% or more than about 90%.

“Ganglioside replacement therapy”, as this term is used herein refers to a method of administering to a subject in need thereof, a sufficient amount of a ganglioside of the invention, generally as a pharmaceutical formulation thereof, to provide the subject with a positive change in a therapeutically relevant characteristic arising from a disease causing or caused by a deficiency in the ganglioside replace in ganglioside replacement therapy. In exemplary embodiments, ganglioside replacement therapy restores, at least transiently following administration of the replacement ganglioside, the level in the subject of the relevant ganglioside to at least about 90%, about 80%, about 70%, about 60%, about 50%, about 40%, about 30%, about 20%, or at least about 10% of a level considered clinically normal for a subject not affected by the disease. In exemplary embodiments, one or more clinically relevant characteristics of the subject attributable to the disease are resolved or reduced, at least transiently following administration of the replacement ganglioside, by at least about 90%, about 80%, about 70%, about 60%, about 50%, about 40%, about 30%, about 20%, or at least about 10% of the level of the characteristic in the patient prior to ganglioside replacement therapy. In an exemplary embodiment, ganglioside replacement therapy utilizes a precursor to the one or more gangliosides to be replaced, the subject converting at least a fraction of the administered precursor to the one or more gangliosides to be replaced by the therapy.

In an exemplary embodiment, a pharmaceutical formulation of the invention comprises a therapeutically effective amount of the GM1 of the invention. As used herein, “therapeutically effective amount” or “an amount effective” refers to an amount of the pharmaceutical formulation of the invention which is effective, upon single or multiple dose administrations to a subject, in treating a cell, or curing, alleviating, relieving or improving a symptom of a disorder. An effective amount of the composition may vary according to factors such as the disease state, age, sex, and weight of the individual, and the ability of the compound to elicit a desired response in the individual. An effective amount is also one in which any toxic or detrimental effects of the composition are outweighed by the therapeutically beneficial effects.

In various embodiments, the term “therapeutically effective amount” relates to an amount of compound which, when administered according to a desired dosing regimen, provides the desired therapeutic activity. Dosing may occur at intervals of minutes, hours, days, weeks, months or years or continuously over any one of these periods. Exemplary suitable dosages may lie within the range of about 0.1 ng per kg of body weight to 1 g per kg of body weight per dosage, such as is in the range of 1 mg to 1 g per kg of body weight per dosage. In one embodiment, the dosage may be in the range of 1 mg to 500 mg per kg of body weight per dosage. In another embodiment, the dosage may be in the range of 1 mg to 250 mg per kg of body weight per dosage. In yet another embodiment, the dosage may be in the range of 1 mg to 100 mg per kg of body weight per dosage.

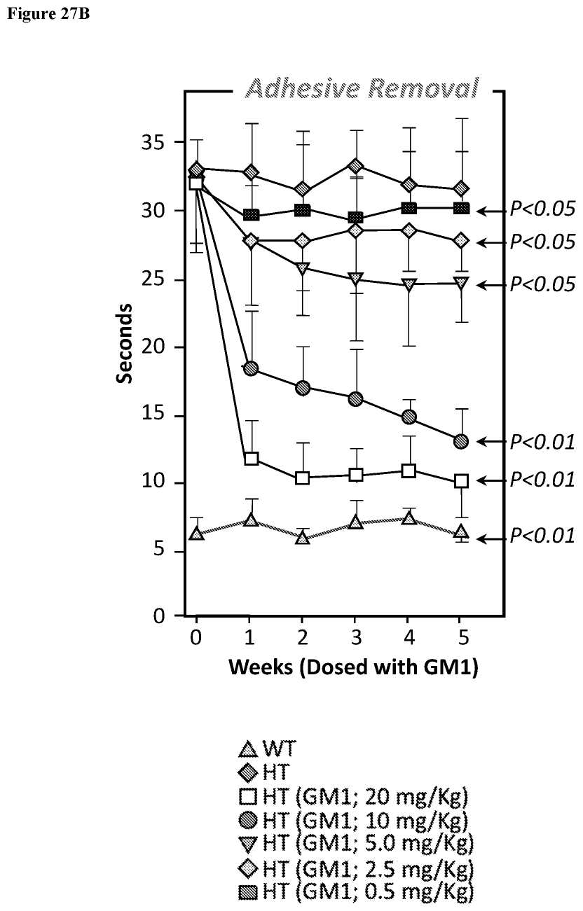

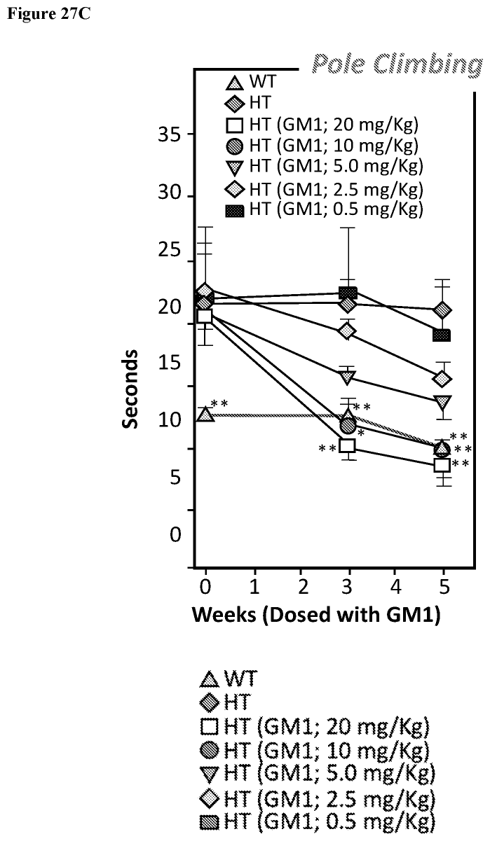

The minimum effective dose of GM1 of the invention was 5 mg/kg (IP, once daily) when administered as ganglioside replacement therapy in ganglioside deficient mice FIG. 27. This dose is capable of reversing motor and cognitive deficits in GM2 and GM3 synthase deficient mice (knock-outs and heterozygotes). The predicted therapeutic human dose based on allometric scaling of all animal and human data is predicted to be 2-10 mg/Kg administered daily as a parenteral (e.g., subcutaneous, intraperitoneal, intramuscular). In an exemplary embodiment, the therapeutically effective amount is from about 2 to about 10 mg per kg of body weight per dosage. In an exemplary embodiment, the dose of the GM1 formulation is administered parenterally once daily.

In an exemplary embodiment, a composition of the invention is of use when administered to a subject in an effective amount to prevent a disease or symptoms of a disease. As used herein, the term “prevent” or “preventing” as used in the context of the administration of an agent to a subject, refers to subjecting the subject to a regimen, e.g., the administration of a pharmaceutical formulation of the invention such that the onset of at least one symptom of the disorder is delayed as compared to what would be seen in the absence of the regimen.

In various embodiments, the compositions of the invention are administered to a subject to treat or prevent a disease or the symptoms of a disease. As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dog, cat, cow, pig, etc.

As used herein, the term “treat” or “treating” a subject having a disorder refers to subjecting the subject to a regimen, e.g., the administration of a pharmaceutical formulation of the invention such that at least one symptom of the disorder is cured, healed, alleviated, relieved, altered, remedied, ameliorated, or improved. Treating includes administering an amount effective to alleviate, relieve, alter, remedy, ameliorate, improve or affect the disorder or the symptoms of the disorder. The treatment may inhibit deterioration or worsening of a symptom of a disorder.

The term “pharmaceutically acceptable salts” includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., Journal of Pharmaceutical Science, 66:1-19 (1977)). Certain specific compounds of the present invention contain both basic and acidic functionalities that allow the compounds to be converted into either base or acid addition salts.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

It will be appreciated that any compound that is a prodrug of the compound the invention is also within the scope and spirit of the invention. Thus the compound of the invention can be administered to a subject in the form of a pharmaceutically acceptable pro-drug. The term “pro-drug” is used in its broadest sense and encompasses those derivatives that are converted in vivo to the compound of the invention. Such derivatives would readily occur to those skilled in the art. Other texts which generally describe prodrugs (and the preparation thereof) include: Design of Prodrugs, 1985, H. Bundgaard (Elsevier); The Practice of Medicinal Chemistry, 1996, Camille G. Wermuth et al., Chapter 31 (Academic Press); and A Textbook of Drug Design and Development, 1991, Bundgaard et al., Chapter 5, (Harwood Academic Publishers). For example, the N atom on the oxindole ring may be reacted with an acid (for example acetic acid). An exemplary pharmaceutically acceptable prodrug is a pharmaceutically acceptable ester.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

The term “solvate” refers to a physical association of one of the present compounds with one or more solvent molecules. This physical association includes hydrogen bonding. In certain instances, the solvate will be capable of isolation, for example when one or more solvent molecules are incorporated in the crystal lattice of a crystalline solid. “Solvate” encompasses both solution-phase and insoluble solvates. Exemplary solvates include, without limitation, hydrates, hemihydrates, ethanolates, hemiethanolates, n-propanolates, iso-propanolates, 1-butanolates, 2-butanolate, and solvates of other physiologically acceptable solvents, such as the Class 3 solvents described in the International Conference on Harmonization (ICH), Guide for Industry, Q3C Impurities: Residual Solvents (1997). The compounds as herein described also include each of their solvates and mixtures thereof.

“Pharmaceutically acceptable excipients”, as used herein, refers to recognized additives in pharmaceutical formulations of active pharmaceutical agents. Exemplary excipients include buffers, salts (e.g., NaCl), sugars, sugar alcohols, and amino acids (e.g., arginine, glycine). They are generally safe for administering to humans according to established governmental standards, including those promulgated by the United States Food and Drug Administration. Representative, non-limiting examples include, agar-agar, algins, calcium carbonate, carboxymethylcellulose, cellulose, gums, low substituted hydroxypropylcellulose, sodium starch glycolate, carbonate, calcium phosphate, dibasic calcium phosphate, tribasic calcium sulfate, calcium carboxymethylcellulose, cellulose, dextrin derivatives, dextrin, dextrose, fructose, lactitol, lactose, magnesium carbonate, magnesium oxide, maltitol, maltodextrins, maltose, sorbitol, starch, sucrose, sugar, xylitol, calcium stearate, ethyl oleate, ethyl laureate, glycerin, glyceryl palmitostearate, mannitol, poloxamer, glycols, sodium benzoate, and sodium lauryl sulfate.

Certain compounds of the present invention possess asymmetric carbon atoms (optical centers) or double bonds; the racemates, diastereomers, geometric isomers and individual isomers are encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The expression “pharmaceutically acceptable carrier, adjuvant, or vehicle” and equivalent expressions, refer to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester, prodrug, salt of a prodrug, or other derivative of a compound of the present description that, upon administration to a recipient, is capable of providing, either directly or indirectly, a compound of the present description or an inhibitory active metabolite or residue thereof.

### The Compositions

The present invention provides highly pure ganglioside preparations, methods of using the gangliosides in these preparations in therapeutic modalities and methods of making these compounds. Also provided are pharmaceutically acceptable derivatives, prodrugs, salts, solvates and formulations of the highly pure gangliosides of the invention. In some embodiments, methods of preparing the highly pure ganglioside compounds of the invention are provided.

In one embodiment, the invention provides a ganglioside composition in which the sphingosine moiety of the ganglioside is present in a purity of at least 96%, at least about 97%, at least about 98% or at least about 99%. The compositions of the invention include a ganglioside fraction in which more than about 96% of the sphingosine moiety present is in a single form, i.e., the composition comprises a ganglioside fraction in which a single form of sphingosine is present in at least about 96% abundance, at least about 97% abundance, at least about 98% abundance or at least about 99% abundance. In an exemplary embodiment, the single form is a single stereoisomer. In an exemplary embodiment, the ganglioside is GM1.

In an exemplary embodiment, the content of each contaminant in the ganglioside composition of the invention is not more than about 0.2%. In various embodiments, the content of each contaminant in the composition is not more than about 0.1%.

In an exemplary embodiment, the invention provides a ganglioside composition having a ganglioside fraction in which sphingosine is present in the ganglioside as a substantially pure stereoisomer, the composition having at least 98%, at least about 99% of a single stereoisomer. In an exemplary embodiment, the ganglioside is GM1.

In an exemplary embodiment, the content of each contaminant in the GM1 composition of the invention is not more than about 0.2%. In various embodiments, the content of each contaminant in the GM1 composition is not more than about 0.1%.

In an exemplary embodiment, the invention provides a ganglioside composition in which a substantial fraction of the saccharide population does not include a (Gal)ₙ-moiety attached to the GalNAc of the ceramide Gal-GalNAc-(Sia)-Gal-Glu saccharide. The index n is an integer of 1 or greater. Thus, the invention provides a ganglioside composition in which the improvement is the absence of a (Gal)ₙ moiety from the terminal Gal of the ceramidyl saccharide Gal-GalNAc-(Sia)-Gal-Glu. In various embodiments, the (Gal)ₙ moiety is absent from at least about 99% of the ganglioside, at least about 98%, at least about 96%, at least about 92%, at least about 90%, at least about 85% or at least about 80% of the ganglioside.

In an exemplary embodiment, the ganglioside composition from which the (Gal)ₙ moiety is substantially absent from the terminal Gal of the ceramidyl saccharide Gal-GalNAc-(Sia)-Gal-Glu is a GM1 preparation.

In various embodiments, the ganglioside composition is GM1. The sphingosine d18:1 content in the GM1 of the composition is at least about 96%, at least about 97%, at least about 98%, or at least about 99% of the total weight of the GM1 in the composition.

An exemplary embodiment of the GM1 formulation has a GM1 fraction with the following characteristics:
Sphingosine (d18:1)—at least about 96%; and a member selected from:
   Sphingosine (d17:1)—no more than about 0.1%;
   Sphinganine (d18:0)—no more than about 2%;
   Sphinganine (d20:0 —NH₂)—no more than about 0.5%;
   Sphingosine (d22:1—NH₂)—no more than about 0.5%,
      and a combination thereof;
Stearic acid (C18:0)—at least about 92%; and a member selected from:
   Palmitic acid (C16:0)—no more than about 3%;
   Margaric acid (C17:0)—no more than about 1%;
   Nonadecylic acid (C19:0)—no more than about 0.2%;
   Arachidic acid (C20:0)—no more than about 2%
      and a combination thereof.

In an exemplary embodiment, the fraction described above is at least about 80%, at least about 85%, at least about 90%, at least about 92%, at least about 94%, at least about 96%, at least about 98%, or at least about 90% of the total weight of the GM1 in the composition.

An exemplary embodiment of the GM1 composition of the invention comprises a GM1 fraction having the following characteristics:
Sphingosine (d20:1)—at least about 96%; and a member selected from:
   Sphinganine (d20:0)—no more than about 2.5%;
   Sphingosine (d18:1)—no more than about 0.4%;
   Sphingosine (d16:1)—no more than about 0.4%;
   Sphingosine (d22:1)—no more than about 2%;
      and a combination thereof;
Stearic acid (C18:0)—at least about 92%; and a member selected from:
   Palmitic acid (C16:0)—no more than about 3%;
   Margaric acid (C17:0)—no more than about 1%;
   Nonadecylic acid (C19:0)—no more than about 0.2%;
   Arachidic acid (C20:0)—no more than about 2%
      and a combination thereof.

An exemplary embodiment of the GM1 composition of the invention comprises a GM1 fraction having the following characteristics:
Sphingosine (d19:1)—at least about 96%; and a member selected from:
   Sphinganine (d19:0)—no more than about 2.5%;
   Sphingosine (d18:1)—no more than about 0.4%;
   Sphingosine (d17:1)—no more than about 0.4%;
   Sphinganine (d18:0)—no more than about 2%;
   Sphinganine (d20:0)—no more than about 0.5%;
   Sphingosine (d20:1)—no more than about 0.5%,
      and a combination thereof;
Stearic acid (C18:0)—at least about 92%; and a member selected from:
   Palmitic acid (C16:0)—no more than about 3%;
   Margaric acid (C17:0)—no more than about 1%;
   Nonadecylic acid (C19:0)—no more than about 0.2%;
   Arachidic acid (C20:0)—no more than about 2%
      and a combination thereof.

An exemplary embodiment of the GM1 composition of the invention comprises a GM1 fraction having the following characteristics:
Sphingosine (d17:1)—at least about 96%; and a member selected from:
   Sphinganine (d17:0)—no more than about 2.5%;
   Sphingosine (d18:1)—no more than about 0.4%;
   Sphingosine (d16:1)—no more than about 0.4%;
   Sphinganine (d18:0)—no more than about 2%;
   Sphinganine (d19:0)—no more than about 0.5%;
      and a combination thereof;
Stearic acid (C18:0)—at least about 92%; and a member selected from:
   Palmitic acid (C16:0)—no more than about 3%;
   Margaric acid (C17:0)—no more than about 1%;
   Nonadecylic acid (C19:0)—no more than about 0.2%;
   Arachidic acid (C20:0)—no more than about 2%
      and a combination thereof.

An exemplary embodiment of the GM1 composition of the invention comprises a GM1 fraction having the following characteristics:
Sphingosine (d19:1)—at least about 96%; and a member selected from:
   Sphinganine (d19:0)—no more than about 2.5%;
   Sphingosine (d18:1)—no more than about 0.4%;
   Sphingosine (d20:1)—no more than about 0.4%;
   Sphinganine (d18:0)—no more than about 2%;
   Sphinganine (d20:0)—no more than about 0.5%;
      and a combination thereof;
Stearic acid (C18:0)—at least about 92%; and a member selected from:
   Palmitic acid (C16:0)—no more than about 3%;
   Margaric acid (C17:0)—no more than about 1%;
   Nonadecylic acid (C19:0)—no more than about 0.2%;
   Arachidic acid (C20:0)—no more than about 2%
      and a combination thereof.

An exemplary embodiment of the GM1 composition of the invention comprises a GM1 fraction having the following characteristics:
Sphingosine (d18:1)—at least about 96%; and a member selected from:
   Sphingosine (d17:1)—no more than about 0.1%;
   Sphinganine (d18:0)—no more than about 2%;
   Sphinganine (d20:0 —NH₂)—no more than about 0.5%;
   Sphingosine (d22:1 —NH₂)—no more than about 0.5%,
      and a combination thereof;
Stearic acid (C18:0)—at least about 92%; and a member selected from:
   Palmitic acid (C16:0)—no more than about 3%;
   Margaric acid (C17:0)—no more than about 1%;
   Nonadecylic acid (C19:0)—no more than about 0.2%;
   Arachidic acid (C20:0)—no more than about 2%
   and a combination thereof;
Sphingosine (d18:1) chirality—at least about 96% D-erythro- and a member selected from:
   Sphingosine (d18:1) chirality—no more than about 0.5% of D-threo-;
   Sphingosine (d18:1) chirality—no more than about 0.5% L-threo-;
   Sphingosine (d18:1) chirality—no more than about 0.5% L-erythro-;
      and a combination thereof.

An exemplary embodiment of the GM1 composition of the invention comprises a GM1 fraction having the following characteristics:
Sphingosine (d20:1)—at least about 96%; and a member selected from:
   Sphinganine (d20:0)—no more than about 2.5%;
   Sphingosine (d18:1)—no more than about 0.4%;
   Sphingosine (d16:1)—no more than about 0.4%;
   Sphingosine (d22:1)—no more than about 2%;
      and a combination thereof;
Stearic acid (C18:0)—at least about 92%; and a member selected from:
   Palmitic acid (C16:0)—no more than about 3%;
   Margaric acid (C17:0)—no more than about 1%;
   Nonadecylic acid (C19:0)—no more than about 0.2%;
   Arachidic acid (C20:0)—no more than about 2%
      and a combination thereof;
Sphingosine (d20: 1) chirality-at least about 96% D-erythro- and a member selected from:
   Sphingosine (d20:1) chirality—no more than about 0.5% of D-threo-;
   Sphingosine (d20:1) chirality—no more than about 0.5% L-threo-;
   Sphingosine (d20:1) chirality—no more than about 0.5% L-erythro-;
      and a combination thereof.

In an exemplary embodiment, the fraction described above is at least about 80%, at least about 85%, at least about 90%, at least about 92%, at least about 94%, at least about 96%, at least about 98%, or at least about 90% of the total weight of the GM1 in the composition.

In an exemplary embodiment, the fraction described above is at least about 80%, at least about 85%, at least about 90%, at least about 92%, at least about 94%, at least about 96%, at least about 98%, or at least about 99% of the total weight of the GM1 in the composition.

In various embodiments, the invention provides a surprising advance in ganglioside formulations. Gangliosides isolated from natural sources, e.g., GM1, are recognized in the literature as being only sparingly soluble in water, e.g., not more than about 50 mg/mL, unless formulated as a liposome with other more water-soluble components. For example, one preparation of GM1 is disclosed as being soluble to only 3 mg/mL water (Cayman Chemical Product Information, Item No. 19579). Another GM1 preparation from bovine brain is disclosed as only “slightly soluble in water (micellar aggregates)”, (USBiological Life Sciences, G2006-10; CAS: 37758-47-7). Other references describe GM1 as having effectively no solubility in water ((http://www.metabolomicscentre.ca?utm_source=hmdb&utm_medium-banner&utm_campaign=tmic-campaign)).

In contrast to the findings of the literature, exemplary highly pure, structurally homogeneous gangliosides of the invention are readily formulated into highly concentrated, clear, non-cloudy, colorless aqueous solutions. In various embodiments, the concentration of the aqueous formulation of GM1 of the invention is at least about 50 mg/mL. Exemplary formulation concentrations include from about 50 to about 100 mg/mL, about 100 to about 150 mg/mL, about 150 to about 200 mg/mL, about 200 to about 250 mg/mL, about 250 to about 300 mg/mL, about 300 to about 350 mg/mL, about 350 to about 400 mg/mL and about 400 to about 500 mg/mL in GM1. In an exemplary embodiment, the aqueous formulation of GM1 is about 100 mg/mL, about 150 mg/mL, about 200 mg/mL, about 250 mg/mL, or about 300 mg/mL in GM1. In a still more preferred embodiment, the aqueous formulations of GM1 are administered parenterally (i.e., subcutaneous, intraperitoneal, intravenous).

In an exemplary embodiment, the ganglioside solution includes no organic solvent or cosolvent, and the solvent is solely water. In various embodiments, the solutions of the gangliosides show little to no light absorption at 600 nm, indicative of the absence of particles, e.g., micelles, aggregates, precipitates, which are known to form with gangliosides in water. In an exemplary embodiment, not more than about 20%, e.g., 10%, e.g., 5% of light at 600 nm incident on the solution of the invention is absorbed. In various embodiments not more than about 2%, 4%, 6%, 8% or 10% of light at 600 nm incident on the solution of the invention is absorbed. The optical density at 600 nm is a good measure of how turbid or how much scattering material (such as ganglioside micelles, aggregates, precipitates) are in a solution, generally, the more precipitates the higher the absorbance. This is readily determined, e.g., using an available UV/Vis spectrophotometer.

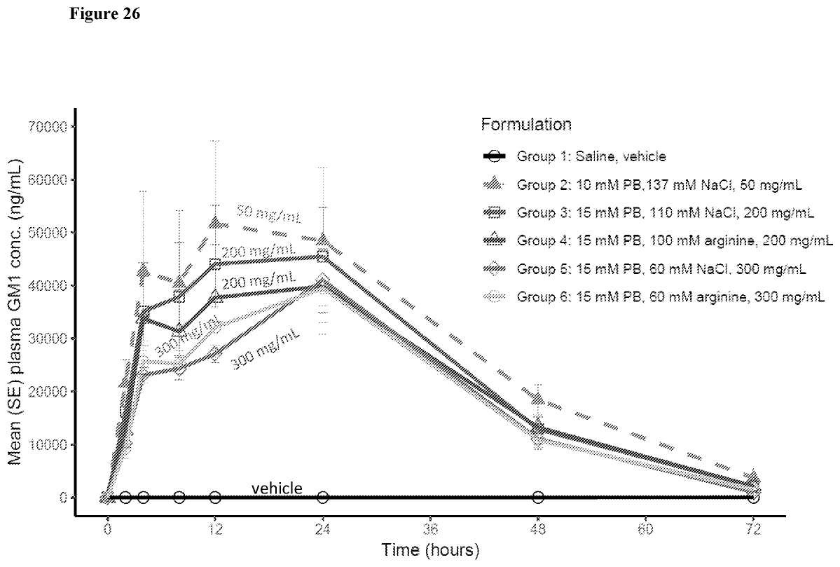

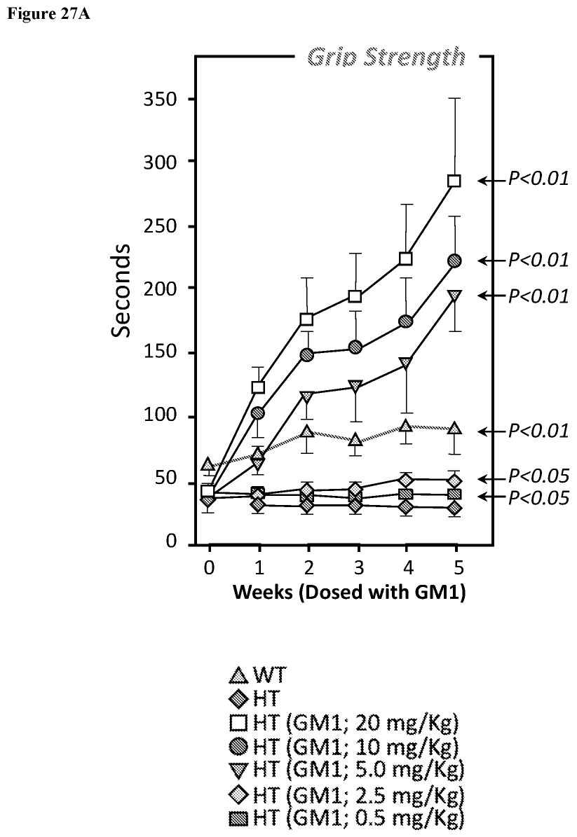

FIG. 26. summarizes exemplary solubility of the GM1 of the invention at various concentrations of GM1, and plasma concentration of the GM1 in Sprague Dawley after subcutaneous administration at the provided concentrations. Formulations of the GM1 of the invention were prepared including GM1 from about 50 mg/mL to about 500 mg/mL in water or buffer with or without excipients, or organic solvents and/or cosolvents.

In various embodiments, the ganglioside preparation of the invention is in the form of a pharmaceutical formulation and is combined with one or more diluent, carrier, additive, excipient, etc. Design of appropriate formulations is within the abilities of those of ordinary skill in the art. In an exemplary embodiment, the ganglioside is GM1, e.g., a GM1 described above.

In various embodiments, the ganglioside composition of the invention is in the form of a pharmaceutical formulation and the ganglioside composition is combined with one or more diluent, carrier, additive, excipient, etc. Design of appropriate formulations is within the abilities of those of ordinary skill in the art. In an exemplary embodiment, the ganglioside is GM1, e.g., a GM1 described above.

In an exemplary embodiment, the ganglioside is in the form of a clear, colorless aqueous pharmaceutical formulation comprising the ganglioside substantially entirely dissolved in water, the formulation optionally comprising one or more additive, or excipient, wherein the formulation is devoid of organic solvents and cosolvents, the formulation comprising from about 50 mg/mL to about 500 mg/mL of the ganglioside (e.g., GM1). In an exemplary embodiment, the ganglioside formulation of the invention is formulated for parenteral administration.

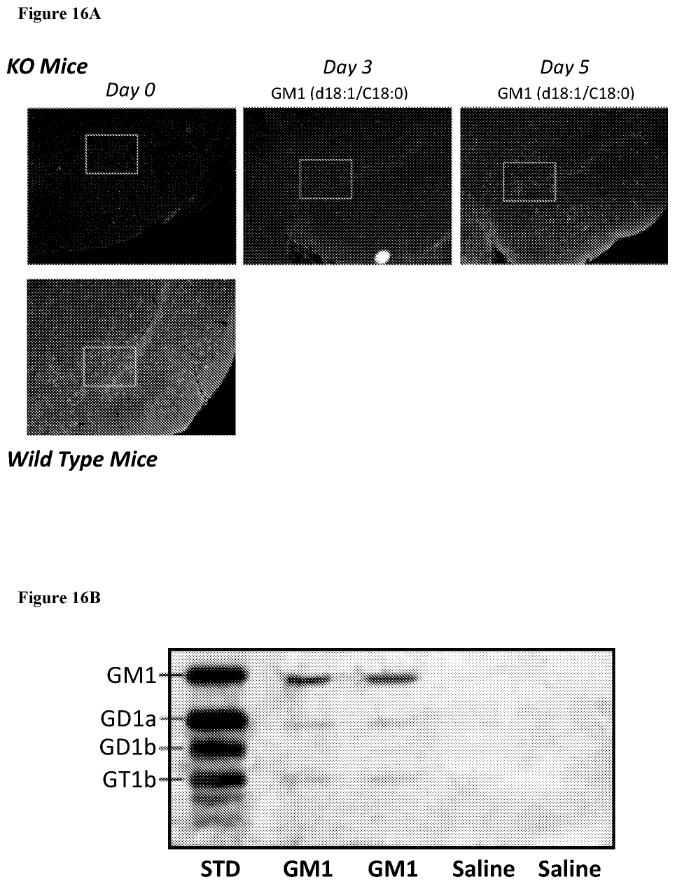

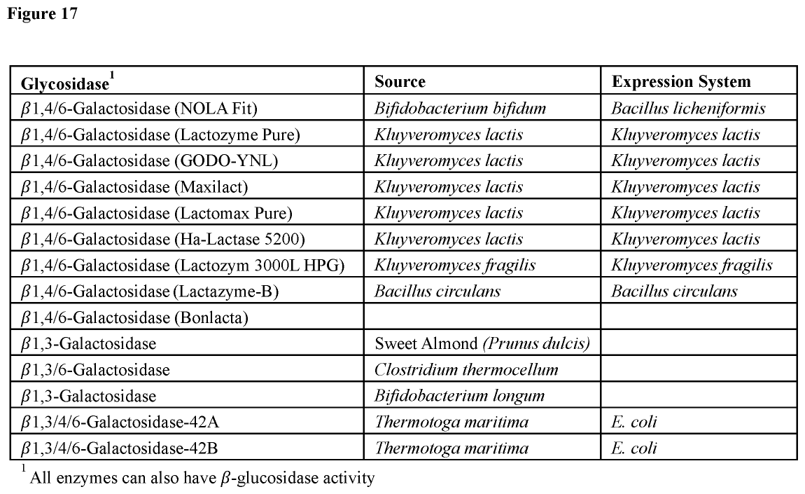

In various embodiments, the ganglioside in the ganglioside preparation of the invention penetrates the blood brain barrier of a mammal. In various embodiments, the ganglioside preparation penetrates the blood brain barrier in the experiment described in FIG. 16, (Wu (2011) Neurochem. Res. 36(9):1706-1714) In an exemplary embodiment, the ganglioside preparation is a preparation of GM1, e.g., a GM1 preparation of the invention.

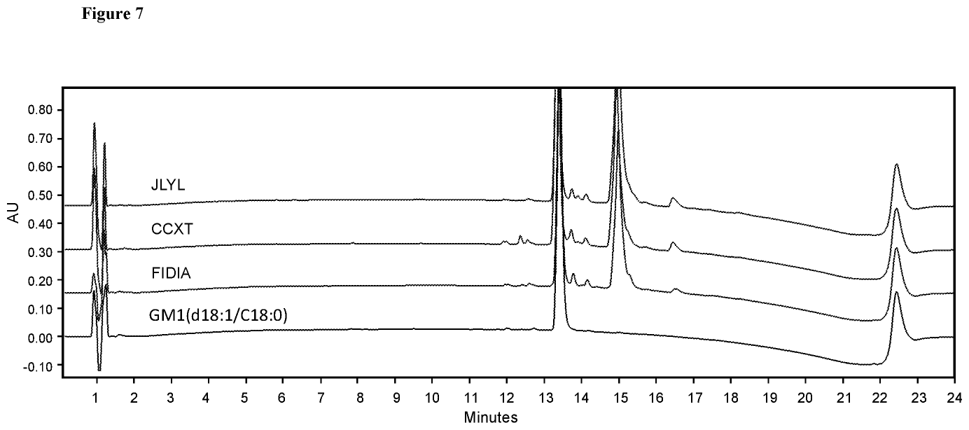

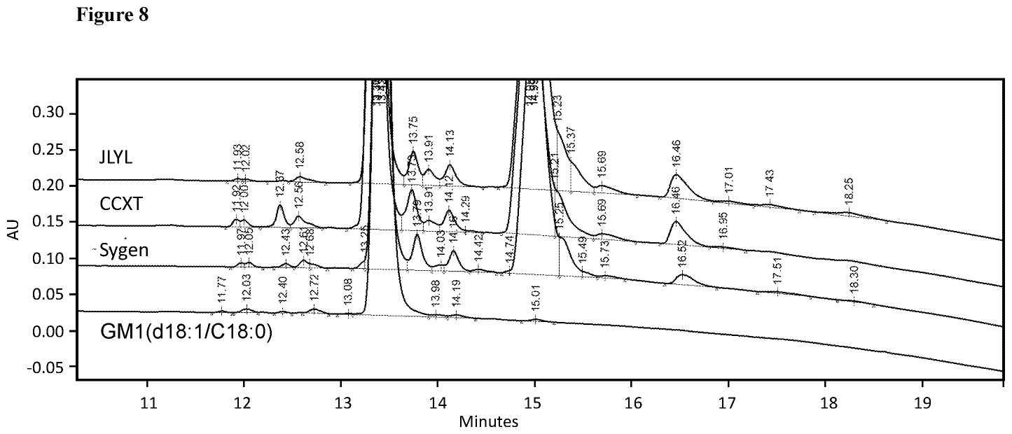

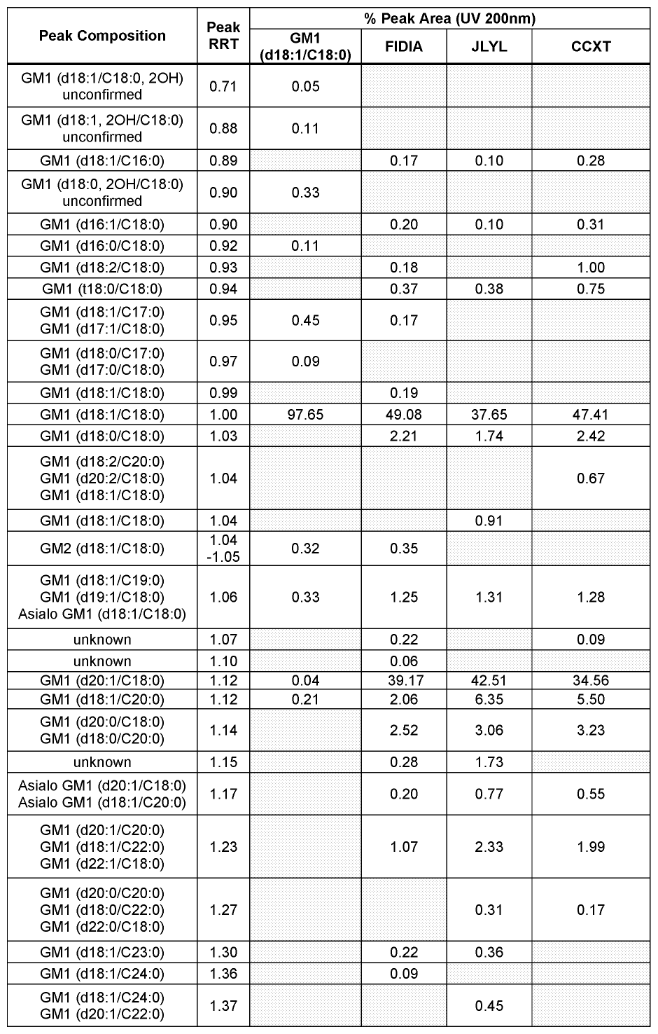

In an exemplary embodiment, the invention provides a GM1 composition, which, when assayed by HPLC according to the conditions set out in Example 3, provides the chromatogram shown in FIG. 7 and expansion FIG. 8.

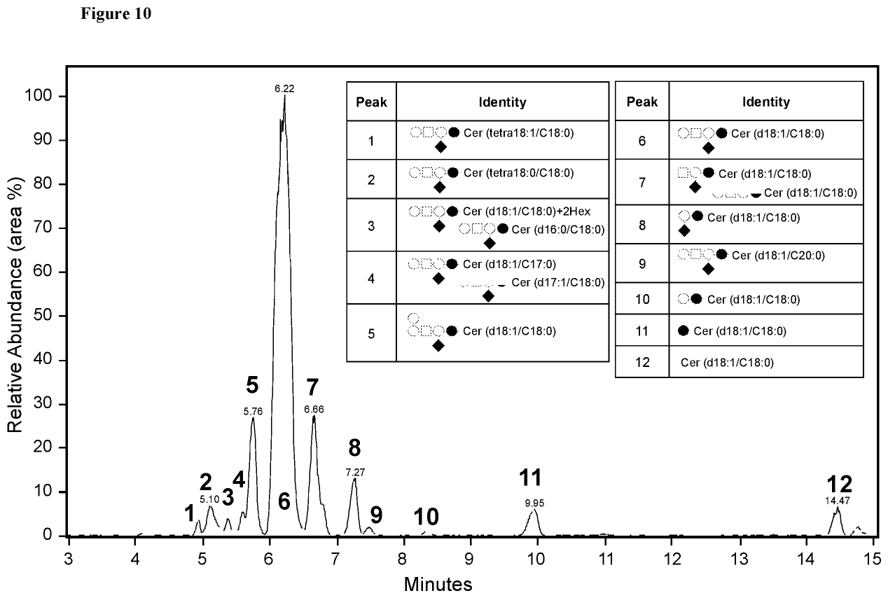

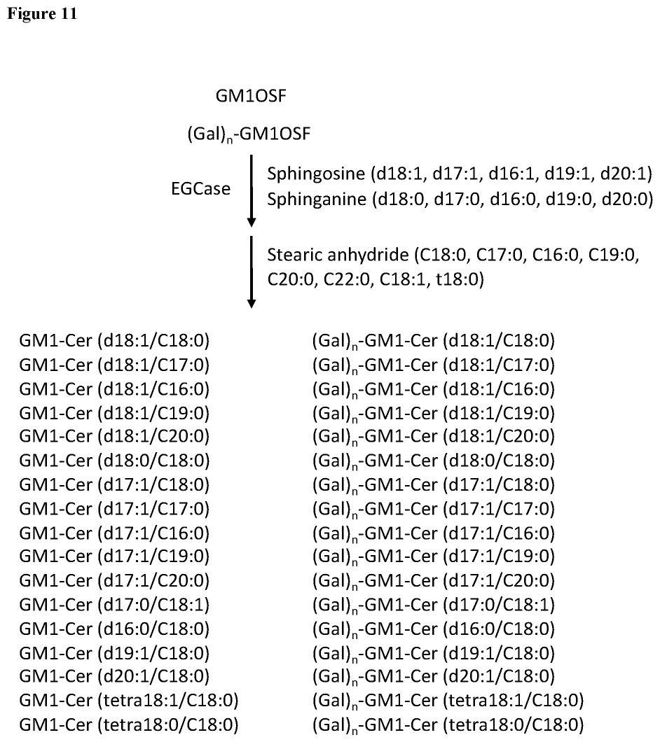

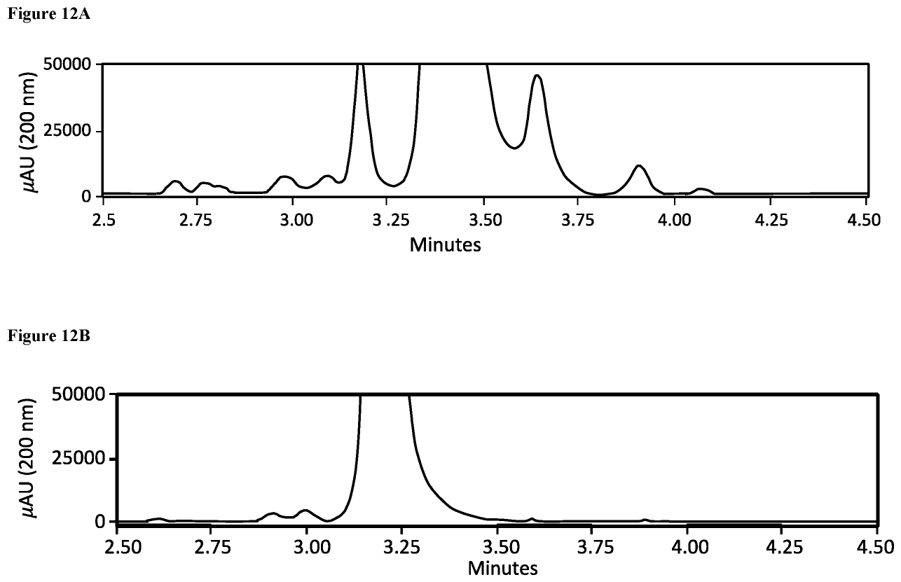

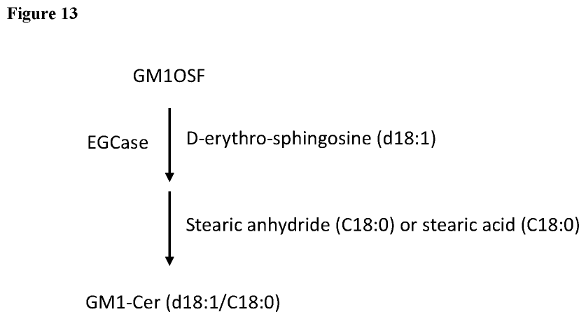

In various embodiments, the invention provides a GM1 composition, which, when assayed by HPLC according to the conditions set forth in Example 3, provides the chromatogram according to FIG. 10 in which one or more of peaks 5, 8, 10, 11 or 12, in any combination, are substantially absent from the chromatogram. In various embodiments, the invention provides a GM1 composition, which, when assayed by HPLC according to the conditions set forth in Example 3, provides the chromatogram according to FIG. 10 in which each of peaks 5, 8, 10, 11 or 12, in any combination, are substantially absent from the chromatogram. In various embodiments, the chromatogram is displayed at a level of magnification at which peak 6 is designated as 100% intensity (relative abundance).

In an exemplary embodiment, the method of the invention provides a ganglioside composition in which the saccharide population does not include a (Gal)ₙ-moiety attached to the GalNAc of the ceramide Gal-GalNAc-(Sia)-Gal-Glu saccharide.

### The Methods

In an exemplary embodiment, the invention provides a method of preparing a highly homogeneous ganglioside composition, e.g., a GM1 composition. In various embodiments, the method is an improved method of preparing a highly homogeneous ganglioside composition, e.g., a GM1 composition, the improvement comprising removing (Gal)ₙ from the terminal Gal of the ceramidyl saccharide Gal-GalNAc-(Sia)-Gal-Glu of the GM1 composition. The index n is an integer of 1 or greater.

Exemplary methods of preparing the ganglioside preparations of the invention involve the enzymatic transfer of carbohydrates, including sialic acids, to a highly pure sphingosine precursor. In particular, the methods involve contacting the pure sphingosine precursor with one or more glycosyltransferases, the corresponding sugar donor moiety for the glycosyltransferases, and other reactants required for glycosyltransferase activity, for a sufficient time and under appropriate conditions to transfer the sugar or sugars from the donor moiety to the sphingosine precursor. In some embodiments, one or more of the enzymatic reactions is carried out in the presence of an organic solvent, which increases the efficiency of the glycosylation reaction.

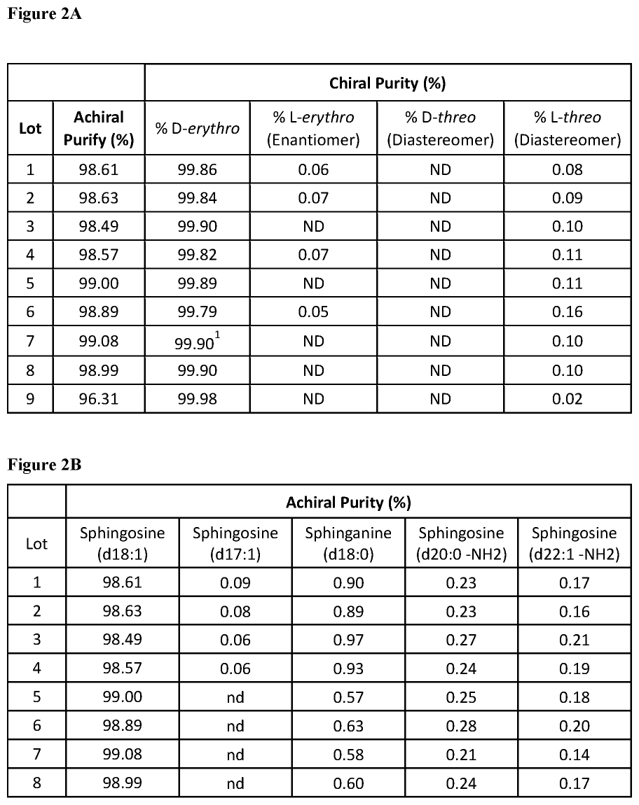

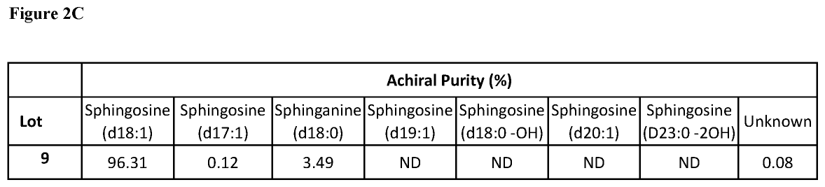

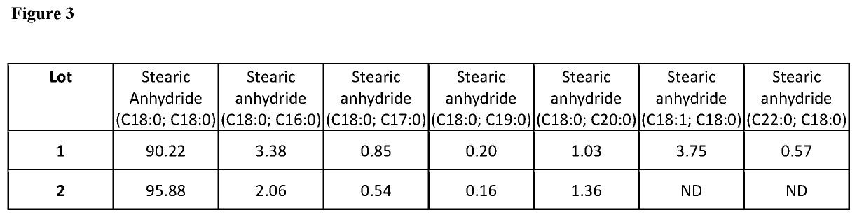

In an exemplary embodiment, the sphingosine is assembled from highly pure precursors, each of which is essentially a single compound. FIG. 2 and FIG. 3. Exemplary precursors have up to about 6% stereoisomeric or other impurity, up to about 5%, up to about 4%, up to about 3%, up to about 2% or up to about 1% of such impurity(ies).

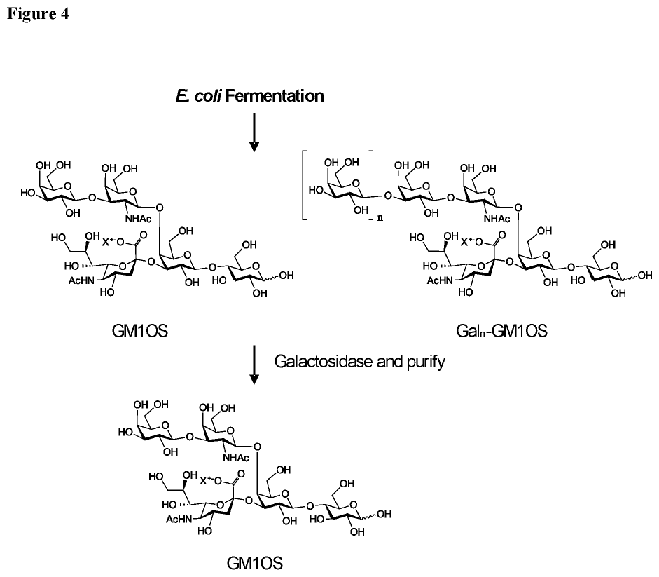

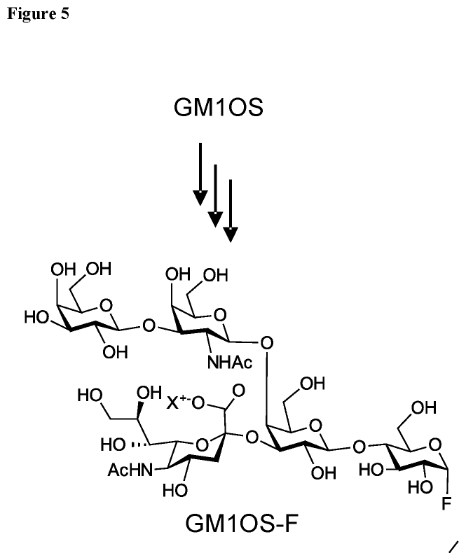

In an exemplary embodiment, the method of the invention includes contacting a ganglioside preparation, thereby removing (Gal)ₙ from the terminal Gal of the ceramidyl saccharide. In an exemplary embodiment, the ganglioside is a GM1 preparation, e.g., a GM1 preparation of the invention, and it is contacted with a galactosidase, thereby converting Galₙ-GM1OS to GM1OS. Exemplary gangliosides of use in this embodiment include those set forth in FIG. 4. In an exemplary embodiment, galactosidase treatment of (Gal)-GM1 produced GM1.

### Uses for Gangliosides and Glycosphingoids

The gangliosides and other compounds that are made using the methods of the invention can be used in a variety of applications, e.g., as antigens, diagnostic reagents, or as therapeutics. For example, gangliosides have been reported to be useful for treating spinal cord and other nervous system injuries (see, e.g., Skaper et al. (1989) Mol. Neurobiol. 3:173; Samson (1990) Drug Devel. Rev. 19: 209-224), stroke, subarachnoid hemorrhage, cognition defects (Kharlamov et al. (1994) Proc. Nat'l. Acad. Sci. USA 91: 6303-6307), Parkinson's disease (Schneider (1998) Ann. N.Y Acad. Sci. 845: 363-73; Schneider (2010) J. Neurol. Sci., 292(1-2):45-51; Schneider (2013) J. Neurol. Sci., 324:140-148), glutamate neurotoxicity (Costa et al. (1994) In Cirrhosis, Hyperammonemia, and Hepatic Encephalopathy, Grisolin and Felipo, Eds., Plenum Press, NY, 1994, p. 129), and other conditions. For review, see, e.g., Nobile-Orazio et al. (1994) Drugs 47:576-585). Gangliosides are involved in the local immunosuppression that is often associated with tumors (Rodden et al. (1991) J. Neurosurg. 74: 606-619), so agents that block or disrupt these gangliosides are useful in reducing the inaccessibility of tumors to the immune system. The immunosuppressive effect of gangliosides is useful for, e.g., suppressing rejection of transplanted organs.

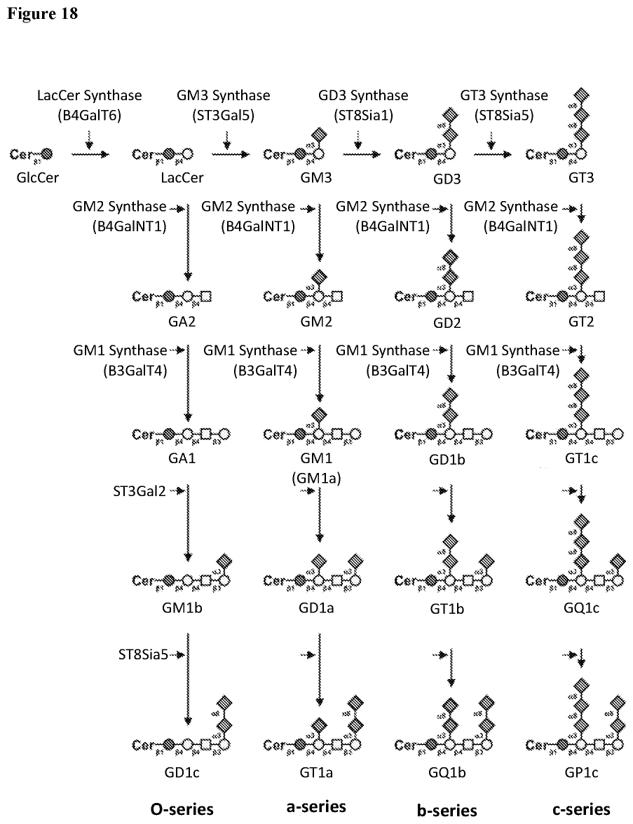

In some embodiment, gangliosides and other compounds made using the methods of the invention can be used as therapeutics to treat diseases that cause a deficiency of one or more ganglioside species, e.g., GM3, GM2 and/or GM1. Any disease may cause a ganglioside deficiency and result from reduced enzyme expression, reduce or missing enzyme activity or enzyme genetic mutation(s) of one or more ganglioside biosynthetic enzymes, FIG. 18. In some embodiments, the ganglioside deficiency is caused by reduced or missing enzyme activity and/or expression selected from GM3 synthase (ST3Gal5), GM2 synthase (B4GalNT1) and GM1 synthase (B3GalT4). In another preferred embodiment, the ganglioside deficiency encompasses reduced amounts or missing ganglioside selected from GM3, GM2 and/or GM1. In a preferred embodiment, the ganglioside level is reduced by 10%, 20%, 30%, 40%, 50%, 60%, 70%, 80%, 90%, or 100% of normal levels. In a more preferred embodiment, the ganglioside level is reduced 40%, 50%, 60%, 70%, 80%, 90% or 100% of normal levels. In a still more preferred embodiment, the ganglioside level is reduced 50%, 60%, 70%, 80%, 90% or 100% compared to normal. In another preferred embodiment, the ganglioside level is reduced 100% compared to normal levels, e.g., the ganglioside is missing. In a most preferred embodiment, the deficient ganglioside is GM1.

In an exemplary embodiment, the method of the invention provides a ganglioside composition that replaces the missing or reduced level of one or more ganglioside(s), a ganglioside replacement therapy. In preferred embodiment, ganglioside compositions of the invention increase the levels of missing or low-level ganglioside by 100%, 90%, 80%, 70%, 60%, 50%, 40%, 30%, 20%, 10% compared to ganglioside levels of the disease state. In a more preferred embodiment, levels of missing or low-level ganglioside are increased by 100%, 90%, 80%, 70%, 60%, 50%, 40% compared to the disease state. In a more preferred embodiment, levels of the missing or low level ganglioside are returned to normal levels, e.g., plus or minus 10%, 20%, or 30% of normal levels. In a preferred embodiment, the missing or low level ganglioside is GM3. In another embodiment, the missing or low level ganglioside is GM2. In a more preferred embodiment, the missing or low-level ganglioside is GM1.

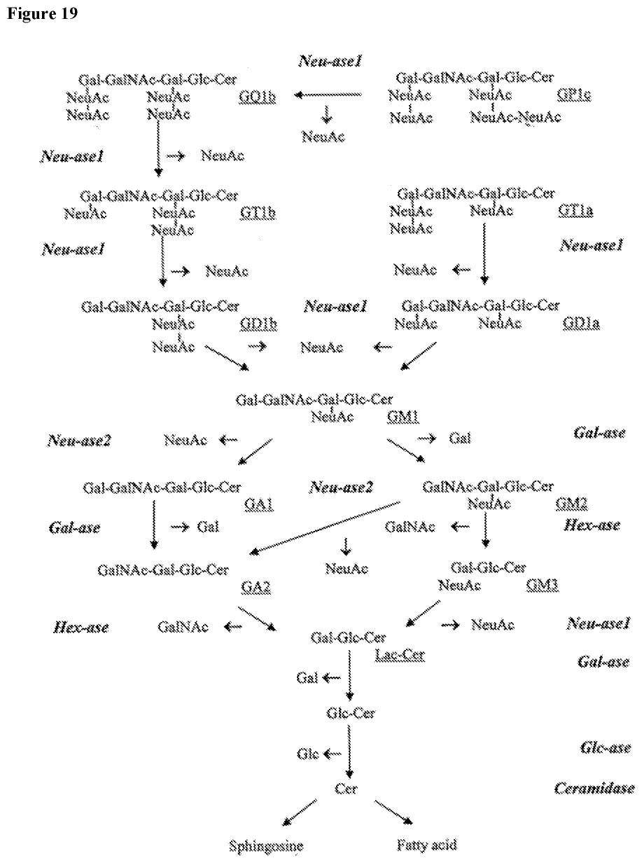

In some embodiments, ganglioside replacement therapy with the ganglioside composition of the invention directly replaces the missing ganglioside, GM1. In other embodiments, ganglioside replacement therapy with the ganglioside composition of the invention also replaces other deficient gangliosides affected by the disease state, e.g., GM3, GM2, GD1a, GT1b and others, FIG. 18. In a preferred embodiment, ganglioside compositions of the invention replace the missing GM1 and are converted to other gangliosides, higher and lower glycoforms, via the ganglioside biosynthetic and salvage pathways, FIGS. 18 and 19 (Riboni (1993) FEBS Letters 322(3):257-260; Ogura (1988) J. Biochem.104(1):87-92;Tettamanti (2003) Biochimie 85(3-4):423-437; Sandhoff (2018) FEBS Letters 592:3835-3864). In preferred embodiment, the ganglioside compositions of the invention are converted to higher ganglioside glycoform selected from GD1a, GT1b, GD1b, GT1a, GQ1a and GQ1b. In still another embodiment, the ganglioside compositions of the invention are converted to lower ganglioside glycoforms selected from GM3, GM2, GD2 and GD3. In a preferred embodiment, the ganglioside compositions of the invention are converted to both higher and lower ganglioside glycoforms encompassing one or more ganglioside species selected from the a-, b- and c-series gangliosides, FIGS. 16B, 18 and 19.

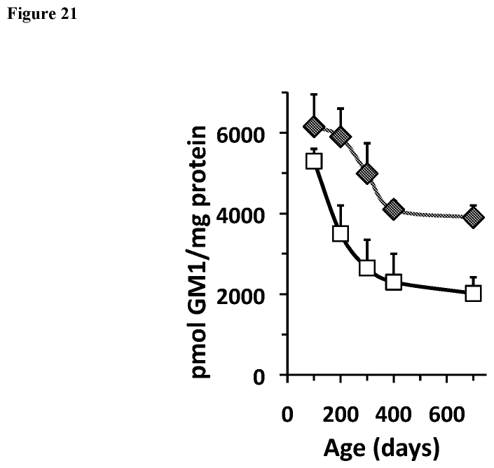

In various embodiments, the ganglioside deficiency is caused by a disease state including diseases caused by gene mutations, e.g. a GM1 deficiency, FIG. 21. For example, Amish Infantile Epilepsy Syndrome (GM3 synthase deficiency; homozygote) is caused by genetic mutation(s) that prevent the production of active GM3 synthase (ST3Gal5), the first step in ganglioside biosynthesis, and prevents the biosynthesis of all a-, b- and c-series gangliosides, FIG. 18 and other glycosphingolipids (Simpson (2004) Nat. Genet. 36(11):1225-1229). Another example, Spastic Paraplesia 26 (GM2 synthase deficiency; homozygote) allows the production of ganglioside GM3, but prevents the production of all higher glycosphingolipid glycoforms that occur after the GM2 synthase step including GM1, FIG. 18 (Boukhris (2013) Am. J. Hum. Genet. 93(1):118-123; Harlalka (2013) Brain 136(Pt 12):3618-3624).

In a further example, the heterozygote (HT) forms of GM3 synthase and GM2 synthase deficiencies, wherein only one functional gene and/or gene product is produced, also develop a progressive ganglioside deficiency (Wu (2011) Neurochem. Res. 36(9):1706-1714). The heterozygote (HT) form of GM3 synthase deficiency causes reduced GM3 and GM1 levels. The heterozygote (HT) form of GM2 synthase deficiency causes reduced GM1 levels with progressive age related loss, FIG. 21 In a preferred embodiment, ganglioside replacement therapy with the ganglioside compositions of the invention replace the missing GM1, and via the salvage and biosynthetic pathways (Riboni (1993) FEBS Letters 322(3):257-260; Ogura (1988) J. Biochem.104(1):87-92; Tettamanti (2003) Biochimie 85(3-4):423-437; Sandhoff (2018) FEBS Letters 592:3835-3864), replaces other missing or low level glycosphingolipids including those selected from GM3, GM2 GD1a and GT1b (FIGS. 16, 18 and 19).

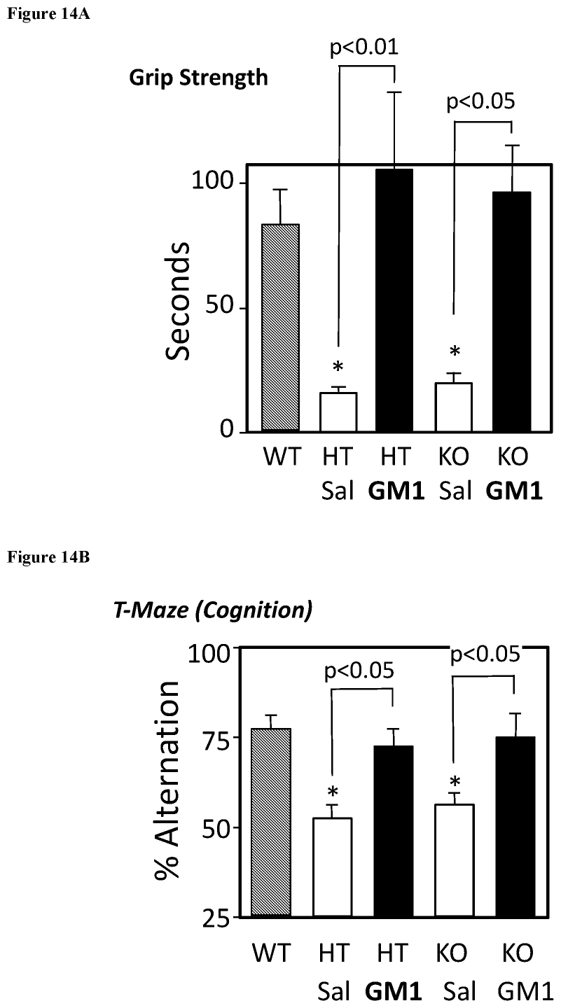

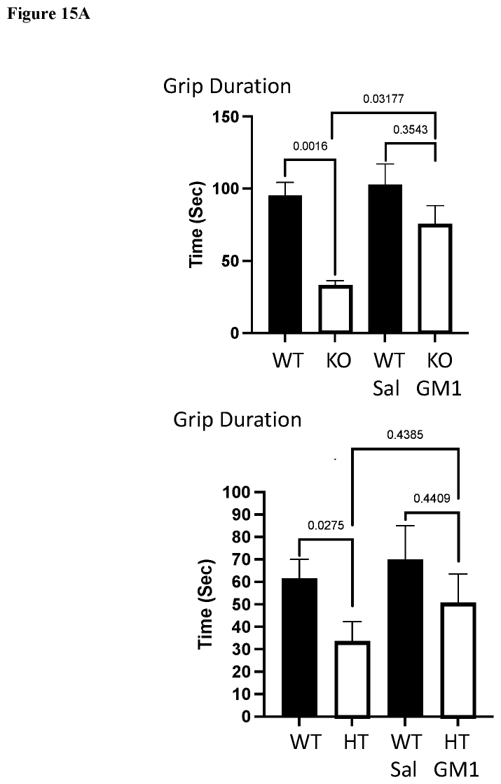

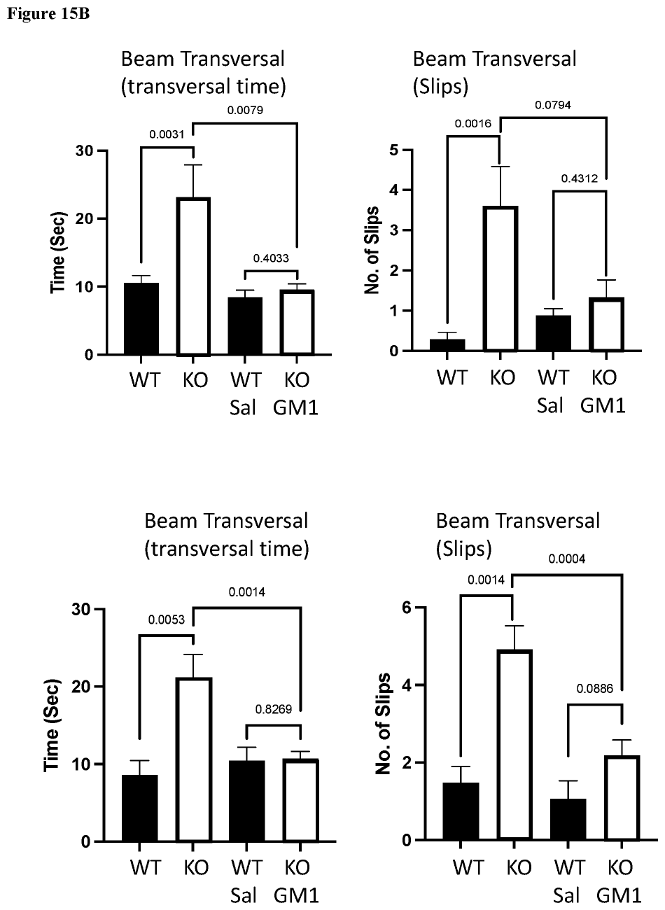

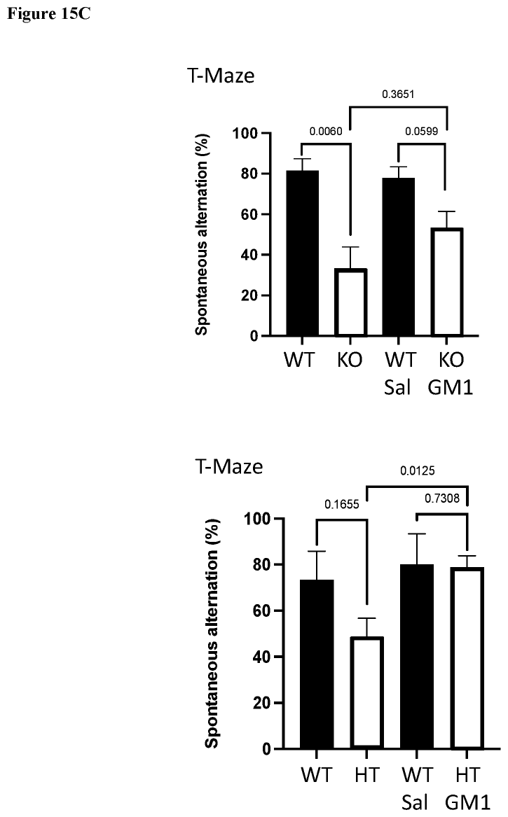

In various embodiments, ganglioside replacement therapy with the ganglioside composition of the invention improves the behavioral abnormalities of subjects and animals with any of the homozygote (KO) or heterozygote (HT) forms of GM3 synthase and GM2 synthase deficiencies. For example, subjects and animals with the homozygote (KO) or heterozygote (HT) forms of GM2 synthase deficiency develop cognitive and motor deficits that can be improved by providing replacement therapy with the ganglioside composition of the invention, FIGS. 14. In another example, subjects and animals with the homozygote (KO) or heterozygote (HT) forms of GM3 synthase deficiency develop cognitive and motor deficits that can be improved by providing replacement therapy with the ganglioside composition of the invention, FIG. 15. In a preferred embodiment, treatment of subjects or animals with the ganglioside composition of the invention improves cognitive function measured by the T-maze (mice) (Wu (2011) Neurochem. Res. 36(9):1706-1714), FIG. 14, or Bayley Scale (subjects) (Armstrong (2010) Chapter 2—The Bayley—III Cognitive Scale, Eds: L. G. Weiss, T. Oakland, G. Aylward, In Practical Resources for the Mental Health Professional, Bayley—III Clinical Use and Interpretation, Academic Press, Pages 29-45) by 10%, 20%, 30%, 40%, 50%, 60%, 70%, 80%, 90% and 100% when compared to normal subjects or animals, e.g., wild type animals. In another preferred embodiment, treatment of subjects or animals with the ganglioside composition of the invention improves motor function measured by grip strength, grip duration, pole climbing, beam transversal, and adhesive tape removal in mice, FIGS. 14 and 15, (Wu (2011) Neurochem. Res. 36(9):1706-1714), or in subjects measured using the Bayley Scale (Armstrong (2010) Chapter 2—The Bayley—III Cognitive Scale, Eds: L. G. Weiss, T. Oakland, G. Aylward, In Practical Resources for the Mental Health Professional, Bayley-III Clinical Use and Interpretation, Academic Press, Pages 29-45) or MDS-UPDRS motor score (Kohat (2021) Front. Neurol. 12:704906) by about 10%, 20%, 30%, 40%, 50%, 60%, 70%, 80%, 90% and 100% compared to normal subjects, the mean baseline motor score of treated subjects or wild type animals. In an exemplary embodiment, the cognitive and/or motor function loss is improved by ganglioside replacement therapy by 60% to 100% compared to healthy subjects or wild type mice. In various embodiments, the cognitive and/or motor function loss is improved by ganglioside replacement therapy by about 70% to about 100% compared to healthy subjects or wild type mice. In an exemplary embodiment, the cognitive and/or motor function loss is improved by about 80% to about 100% compared to normal subjects or wild type mice. In an exemplary embodiment, the cognitive and/or motor function loss is improved by about 90% to about 100% compared to normal subjects or wild type mice. In an exemplary embodiment, ganglioside replacement therapy with compounds of the invention causes a statistically significant improvement in motor function when compared to healthy subjects and animals or untreated subjects or animals. In another exemplary embodiment, ganglioside replacement therapy with compounds of the invention causes a statistically significant improvement in cognitive function when compared to healthy subjects and animals or untreated subjects or animals. In a preferred embodiment, statistically significance is P<0.05. In another embodiment, statistical significance is P<0.01. In yet another embodiment, statistical significance is P<0.005.

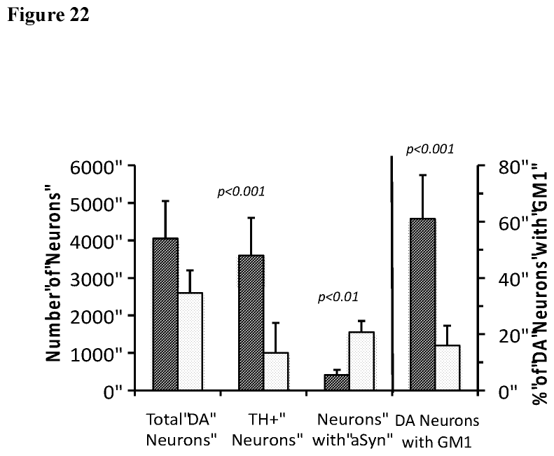

In various embodiments, ganglioside replacement therapy with the ganglioside compositions of the invention replaces a therapeutically relevant amount of missing GM1associated with all forms of Parkinson's disease, e.g., genetic and idiopathic. For example, subjects and animals with idiopathic Parkinson's disease develop a significant reduction in GM1 levels in the CNS, e.g., cortical and dopaminergic neurons, and other cell types and organs, e.g., colon, heart, PBMC's, T-cells, fibroblasts, etc. FIG. 22 (Chowdhury (2022) Biomolecules 12(2):173; Seyfried (2018) ASN Neuro. Jan-Dec; 10:1759091418781889). A reduction in GM1 levels is also observed in genetic forms of Parkinson's disease, for example, a haploinsufficiency of the GBA (glucosylceramidase) and/or GBA2 (glucosylceramidase beta 2) genes (Huebecker (2019) Mol Neurodegener 14(1):40). The reduction in GM1 levels in Parkinson's disease is associated with progressive motor and cognitive function loss in both animals and subjects. Reduced expression of GM2 and/or GM1 synthase in Parkinson's disease neurons correlates with lower GM1 levels and is also associated with disease progression and worsening of symptoms (Chowdhury (2022) Biomolecules 12(2):173; Schneider (2018) PLoS One 13(6):e0199189). In an exemplary embodiment, ganglioside replacement therapy with the ganglioside compositions of the invention replaces the missing GM1 in Parkinson's disease. In some embodiments, ganglioside replacement therapy also replaces other deficient ganglioside levels affected by the disease state, e.g., GM3, GM2, GD1a, GT1b, etc. In some embodiments, the ganglioside compositions of the invention replace the missing GM1 and are converted to other gangliosides, higher and lower glycoforms, via the ganglioside salvage pathway (Riboni (1993) FEBS Letters 322(3):257-260; Ogura (1988) J. Biochem.104(1):87-92; Tettamanti (2003) Biochimie 85(3-4):423-437; Sandhoff (2018) FEBS Letters 592:3835-3864).

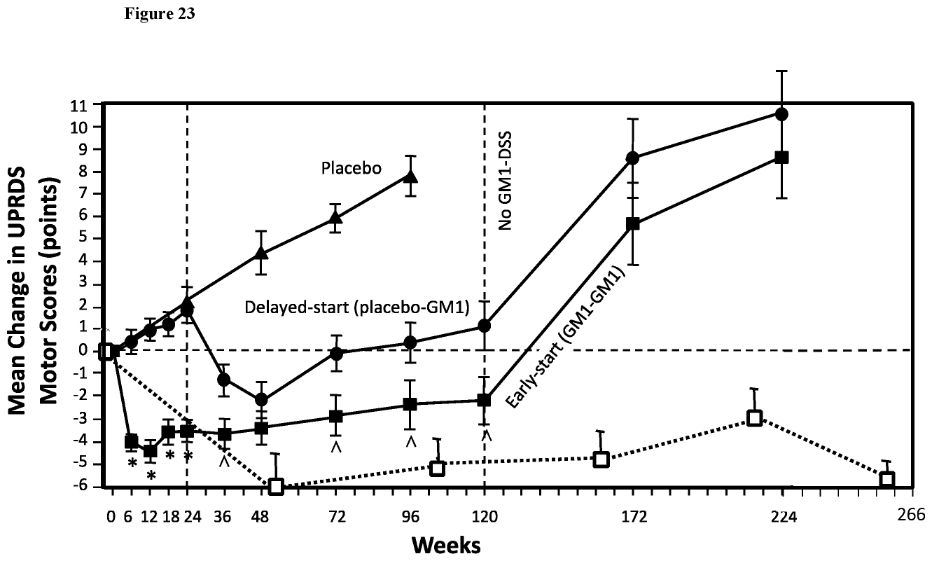

In various embodiments, ganglioside replacement therapy with the ganglioside compositions of the invention improves the behavioral deficits of subjects and animals with any form of Parkinson's disease. For example, subjects and animals with idiopathic or genetic forms of Parkinson's disease develop cognitive and motor deficits that can be improved by providing replacement therapy with the ganglioside compositions of the invention, FIG. 23. In a preferred embodiment, treatment of subjects and animals with the ganglioside composition of the invention improves cognitive function measured in mice by the T-maze (Wu (2011) Neurochem. Res. 36(9):1706-1714), or in subjects by the MDS-UPDRS function score (Kohat (2021) Front. Neurol. 12:704906) by 10%, 20%, 30%, 40%, 50%, 60%, 70%, 80%, 90% and 100% when compared to normal subjects or animals, e.g., wild type animals. In another preferred embodiment, treatment of subjects and animals with the ganglioside composition of the invention improves motor function measured in mice by grip strength, pole climbing, and adhesive tape removal (Wu (2011) Neurochem. Res. 36(9):1706-1714), or the in subjects using the MDS-UPDRS motor function score (Kohat (2021) Front. Neurol. 12:704906) by 5%, 10%, 20%, 30%, 40%, 50%, 60%, 70%, 80%, 90% and 100% compared to the motor and/or cognitive function score of untreated Parkinson's disease subjects or wild type animals. In a preferred embodiment, treatment of subjects and animals with the ganglioside compositions of the invention improves cognitive and/or motor function by 5% to 30%, 30% to 50%, 50% to 70% or 70% to 100% compared to the motor function score of untreated Parkinson's disease subjects or wild type animals. In another preferred embodiment, treatment of subjects with the ganglioside compositions of the invention improves the mean change in UPDRS motor score by −1, −2, −3, −4, −5, −6, −7, −8, −9, or −10 points compared to the baseline motor function score of untreated Parkinson's disease subjects, FIG. 23. In an exemplary embodiment, the mean change in UPDRS motor score is between −2 to −6 points.

In a further preferred embodiment, treatment of subjects and animals with the ganglioside compositions of the invention prevents the annual mean change in UPDRS motor score by 0, 1, 2, 3, or 4 points compared to the previous year mean motor function score of treated Parkinson's disease subjects. In an exemplary embodiment, the annual mean change in UPDRS motor score is 0 to 1 points compared to the previous year mean motor function score of treated Parkinson's disease subjects.

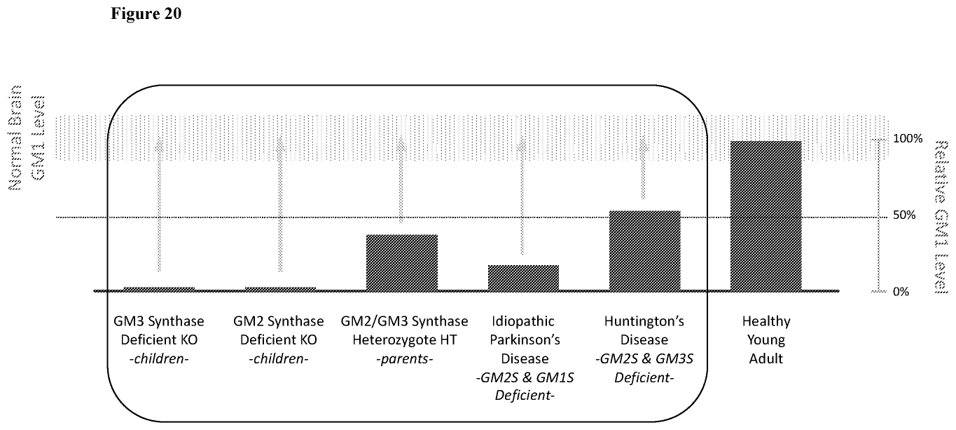

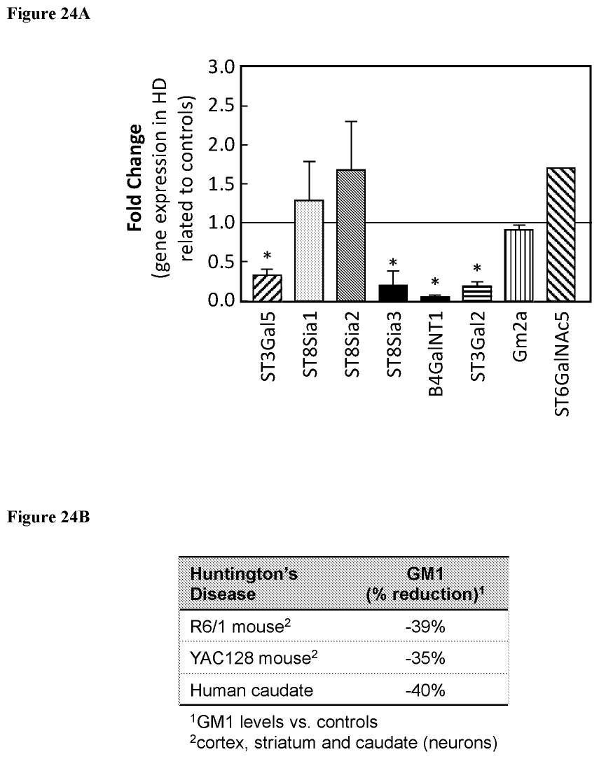

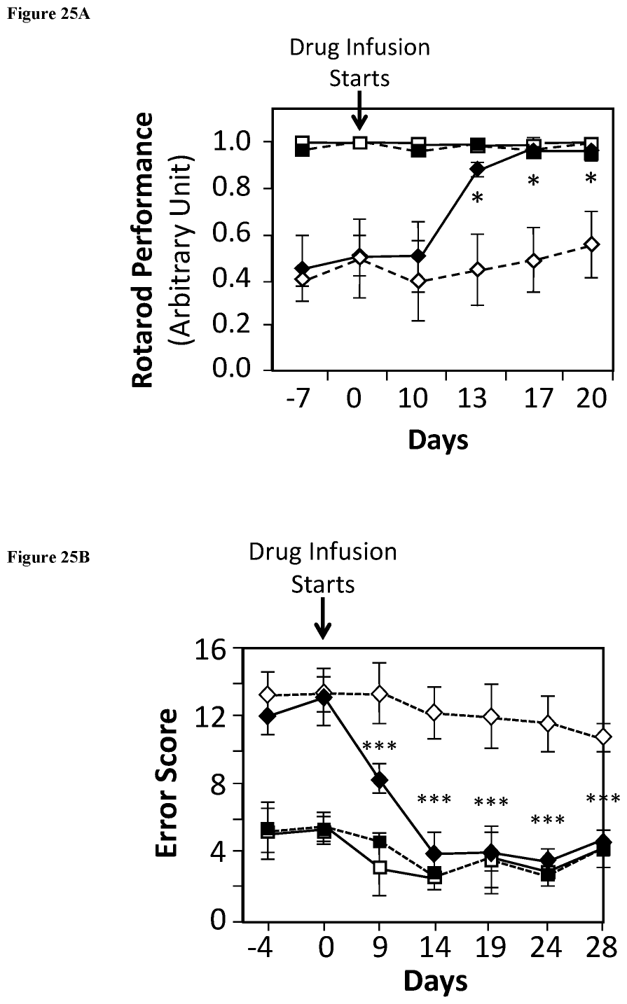

In still other embodiments, gangliosides and other compounds of the invention can be used as a therapeutic to treat Huntington's disease caused by a deficiency of one or more ganglioside species, e.g., GM3, GM2 and/or GM1, FIGS. 20 and 24 (Di Pardo (2016) Front. Neurosci. 10:457). In some embodiments, the ganglioside deficiency is caused by reduced or missing enzyme activity and/or expression selected from GM3 synthase (ST3Gal5), GM2 synthase (B4GalNT1) and GM1 synthase (B3GalT4), FIG. 24 (Desplats (2007) Neurobiol. Dis. 27(3): 265-277). In another preferred embodiment, the ganglioside deficiency encompasses reduced amounts or missing ganglioside selected from GM3, GM2 and/or GM1. In a preferred embodiment, ganglioside replacement therapy with the ganglioside composition of the invention improves the behavioral abnormalities of Huntington's disease subjects and animals, FIG. 25. For example, Huntington's disease subjects and animals with develop cognitive and motor deficits that can be improved by providing replacement therapy with the ganglioside composition of the invention, FIG. 25 (Di Pardo (2012) PNAS 109:3528-3533); Alpaugh (2017) EMBO Molecular Medicine 9(11):1537-1557). In another preferred embodiment, the ganglioside and other compounds of the invention are administered by injection, e.g., intraperitoneal, intrathecal and intracerebral ventricular administration.

Thus, the present invention also provides pharmaceutical formulations which can be used in treating a variety of conditions. The pharmaceutical compositions include the gangliosides or glycosphingoids synthesized using the methods of the invention, along with a pharmaceutically acceptable carrier. Pharmaceutical compositions of the invention are suitable for use in a variety of drug delivery systems. Suitable formulations for use in the present invention are found in Remington's Pharmaceutical Sciences, Mace Publishing Company, Philadelphia, Pa., 17th ed. (1985). For a brief review of methods for drug delivery, see, e.g., Langer, Science 249:1527-1533 (1990).

The pharmaceutical compositions are intended for parenteral, intranasal, topical, oral or local administration, such as by aerosol or transdermally, for prophylactic and/or therapeutic treatment. Commonly, the pharmaceutical compositions are administered parenterally, e.g., intravenously. Thus, the invention provides compositions for parenteral administration which comprise the compound dissolved or suspended in an acceptable carrier, preferably an aqueous carrier, e.g., water, buffered water, saline, PBS and the like. The compositions may contain pharmaceutically acceptable auxiliary substances as required to approximate physiological conditions, such as pH adjusting and buffering agents, tonicity adjusting agents, wetting agents, detergents and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9 and most preferably from 7 and 8.

In some embodiments the gangliosides and other glycosphingoids made using the invention can be incorporated into liposomes formed from standard vesicle-forming lipids. A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028. The targeting of liposomes using a variety of targeting agents (e.g., the oligosaccharide moieties of the gangliosides of the invention) is well known in the art (see, e.g., U.S. Pat. Nos. 4,957,773 and 4,603,044).

The compositions containing the gangliosides and other glycosphingoids can be administered for prophylactic and/or therapeutic treatments. In therapeutic applications, compositions are administered to a patient already suffering from a disease, as described above, in an amount sufficient to cure or at least partially arrest the symptoms of the disease and its complications. An amount adequate to accomplish this is defined as a “therapeutically effective dose.” Amounts effective for this use will depend on the severity of the disease and the weight and general state of the patient, but generally range from about 0.5 mg to about 40 g of oligosaccharide per day for a 70 kg patient, with dosages of from about 5 mg to about 20 g of the compounds per day being more commonly used.

Single or multiple administrations of the compositions can be carried out with dose levels and pattern being selected by the treating physician. In any event, the pharmaceutical formulations should provide a quantity of the oligosaccharides of this invention sufficient to effectively treat the patient.

The gangliosides and other glycosphingoids may also find use as diagnostic reagents. Diagnostic reagents that contain gangliosides made by the methods of the invention, or moieties that bind to the specific gangliosides (e.g., lectins and antibodies), are useful in diagnosing several conditions, including, for example, Fabry disease (-Gal-Gal-GalCer), Farber disease (ceramides; N-acylsphingosines), Gaucher disease (glucocerebroside), GM1 gangliosidosis (GM1 ganglioside), metachromatic leukodystrophy (sulfatide; cerebroside sulfate), Sandhoff disease (GM2 ganglioside), Tay-Sachs disease (GM2 ganglioside). For this use, the compounds can be labeled with appropriate labels, including radioisotopes such as, for example, ¹²⁵I, ¹⁴C, or tritium.

The gangliosides and other glycosphingoids made using the methods of the invention can be used as an immunogen for the production of monoclonal or polyclonal antibodies specifically reactive with the compounds. The multitude of techniques available to those skilled in the art for production and manipulation of various immunoglobulin molecules can be used in the present invention. Antibodies may be produced by a variety of means well known to those of skill in the art. If desired, the production of antibodies can be enhanced by coupling the ganglioside or other glycosphingolipid to an immunogenic protein (e.g.,KLH) prior to administering the compound to the test animal (see, PCT application PCT/US94/00757, Publ. No. WO 94/16731). Uses for antibodies against gangliosides and other glycosphingolipids include cancer diagnosis and are described in, for example, U.S. Pat. No. 4,887,931.

The production of non-human monoclonal antibodies, e.g., murine, lagomorpha, equine, etc., is well known and may be accomplished by, for example, immunizing the animal with a preparation containing the oligosaccharide of the invention. Antibody-producing cells obtained from the immunized animals are immortalized and screened, or screened first for the production of the desired antibody and then immortalized. For a discussion of general procedures of monoclonal antibody production, see, Harlow and Lane, Antibodies, A Laboratory Manual Cold Spring Harbor Publications, N.Y. (1988).

The following Examples are offered to illustrate, but not to limit, the present invention.

### EXAMPLES

Example 1. Enzymatic synthesis of lyso-GM1 (formula (1) to (19) wherein R is H) by mutant EGC enzymes. Reactions are performed in 25 mM NaOAc (pH 5.0) containing 0.1-0.2% Triton X-100. A typical reaction mixture contained approximately 50 mg/ml of a fluorinated GM1 sugar donor (GM1-F) (Vaughan et al. J Am Chem Soc. 128:6300-6301 (2006); DeFrees, ACS National Meeting, September 10-14 (2006)), 15 mg/mL of an acceptor sphingosine analog, and 2.0 mg/ml of the appropriate EGC mutant in a total reaction volume of 50 μL. Under these conditions, the reaction proceeds to >90% completion within 12 hours at 37°° C. based on HPLC and TLC analysis.

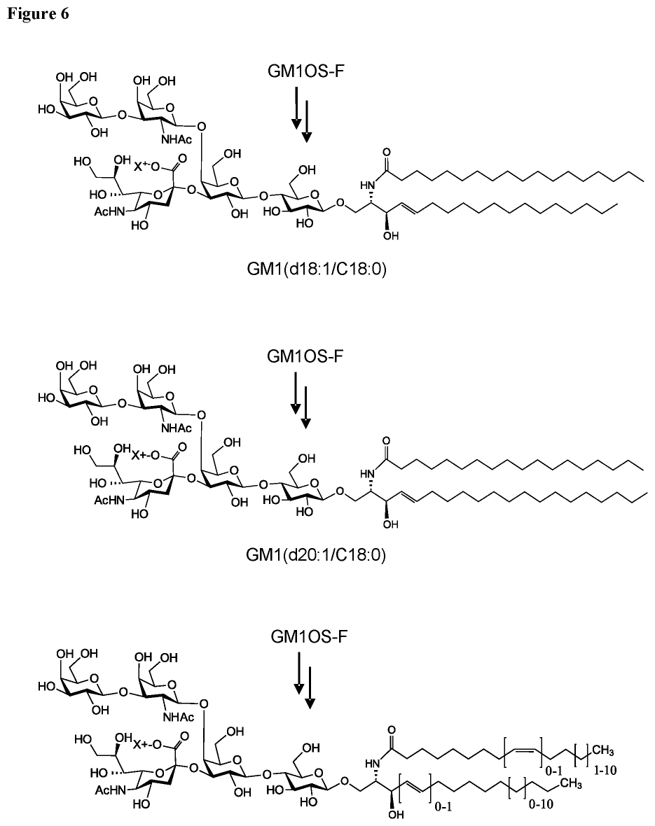

Transfer of the fluorinated GM1 sugar donor is monitored using an HPLC reverse phase method on a Chromolith RP-8e column with eluants of 0.1% trifluoroacetic acid (TFA) in acetonitrile (ACN) to 0.1% TFA in H₂O. The lyso-GM1's are purified using silica gel chromatography. Lyso-GM1 was obtained: TLC (silica gel), (CHCl₃/MeOH/NH₄OH; 4/5/1), R_{f}=0.35 (anisaldehyde); HPLC (RP-8e; acetonitril/water/TFA), Rₜ=2.59 min; ¹H NMR (500 MHz; DMSO-D₂O) (FIG. 6).

Example 2. General Procedure for acylating lyso-GM1. The lyso-GM1 was dissolved in methanol-DMF and triethyl amine (5 mol eq) and fatty acid anhydride or dichloroacetic anhydride (3 mol eq) were added. After stirring overnight, the solution was concentrated to dryness and the acylated GM1 purified by silica gel chromatography. Compound (91) was obtained: TLC (silica gel; CHCl₃/CH₃OH/H₂O/NH₄OH, 5/4/1/0.1), R_{f}=0.62; ¹H NMR (500 MHz, DMSO-D₂O) (FIG. 6).

Example 3. HPLC Method for Analyzing GM1 (d18:1/C18:0). Chromatography was carried out with an Agilent HP Series 1100 liquid chromatographic system (Hewlett Packard, USA). Fitted with an Agilent Poroshell 120 SB-Aq column (2.7 μm, 3.0×150 mm) column. Compound (10 μL) was injected onto a column heated to 40° C. Ultra-high purity nitrogen gas was used as nebulizing gas. The composition of the solvents and gradients for the HPLC-MS/MS method are summarized in Table 1. Mobile phase A was 0.02% TFA in water and Mobile phase B was 0.02% TFA in acetonitrile. Column elution was monitored using UV detection (200 nm).

TABLE 1

| Time (min) | % A | % B |
|---|---|---|
| 0.0 | 65 | 35 |
| 3.0 | 50 | 50 |
| 19.0 | 15 | 85 |
| 20.0 | 15 | 85 |
| 20.1 | 65 | 35 |
| 25.0 | 65 | 35 |

Example 4. Formulation of GM1. Water or buffer solutions were added to GM1 and the suspension stirred until the GM1 was completely dissolved. The solution was a clear, colorless and non-opaque solution. Concentrations of solutions of GM1 prepared by this method included 50 mg/mL, 100 mg/mL, 200 mg/mL, 300 mg/mL, 400 mg/mL and 500 mg/mL. Formulations containing phosphate buffer 0-15 mM (pH 7.0-7.4) were also used and various excipients added including arginine (60-100 mM) and sodium chloride (0-137 mM) (FIG. 26). These formulations were stable at room temperature for months.

Example 5. Pharmacokinetics of Formulated GM1. The formulated GM1 was administered, subcutaneously, to Sprague Dawley rats and the pharmacokinetics determined (FIG. 26). The plasma GM1 levels returned to normal after 72 hours for all GM1 concentrations tested.

The chromatographic columns evaluated in this study included an Atlantis dC18 (2.1×150.0 mm, 3 μm; Waters, USA) and a Discovery HS F5 (2.1×150.0 mm, 3 μm; Supelco, USA). As mobile phases, mixtures of formate buffer (40 mmol L−1, pH 4) and methanol were tested. In all cases, the separation was carried out in a gradient mode with methanol content ranging from 0 to 95%. Mobile phase flow rates from 0.15 to 0.20 mL min−1 were evaluated in accordance with column dimensions and mobile phase composition. Column temperatures between 35 and 45° C. and injection volumes of 3 and 5 L were tested. Detection was carried out at 270 nm for all analytes. The HPLC system UV detector was programmed to make the change in the detection wavelength in the same analytical run, beginning at 233 nm and changing to 270 nm after 7 min.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes to the extent not inconsistent with the present disclosure.

What is claimed is:

1. A ganglioside composition in which at least about 96% of the sphingosine present in the composition is of a single structure.

2. The composition according to claim 1, wherein at least about 96% of the saccharide conjugated to the sphingosine is of a single structure. 3 The composition according to any preceding claim, wherein the sphingosine is sphingosine (d18:1).

4. The composition according to claim 2, wherein the saccharide is Gal-GalNAc-(Sia)-Gal-Glu-.

5. The composition according to any preceding claim, wherein the sphingosine is ceramide.

6. The composition according to any preceding claim, wherein the ganglioside is GM1.

7. The composition according to any preceding claim, wherein the ganglioside is substantially free of a saccharyl moiety of structure: (Gal)ₙ-Gal-GalNAc-(Sia)-Gal-Glu, wherein n is selected from 1 or an integer greater than 1.

8. The composition according to any preceding claim, wherein the ganglioside has the following characteristics:
Sphingosine (d18:1)—at least about 96%; and a member selected from:
Sphingosine (d17:1)—no more than about 0.1%;
Sphinganine (d18:0)—no more than about 2%;
Sphinganine (d20:0 —NH₂)—no more than about 0.5%;
Sphingosine (d22:1—NH₂)—no more than about 0.5%,
and a combination thereof;
Stearic acid (C18:0)—at least about 92%; and a member selected from:
Palmitic acid (C16:0)—no more than about 3%;
Margaric acid (C17:0)—no more than about 1%;
Nonadecylic acid (C19:0)—no more than about 0.2%;
Arachidic acid (C20:0)—no more than about 2%
and a combination thereof.

9. The composition of any preceding claim in the form of a clear, colorless aqueous pharmaceutical formulation comprising the ganglioside substantially entirely dissolved in water, the formulation optionally comprising one or more additive, or excipient, wherein the formulation is devoid of organic solvents and cosolvents, the formulation comprising from about 50 mg/mL to about 500 mg/mL of the ganglioside (e.g., GM1).

10. A method of making the ganglioside composition of any preceding claim, the method comprising:
(a) contacting an essentially pure, essentially structurally homogeneous sphingosine, having no more than about 6% stereoisomeric or other impurity with a first saccharide donor moiety having a first saccharyl moiety and a first glycosyltransferase for which the first saccharide donor moiety is a substrate under conditions appropriate to couple the first saccharyl moiety to the sphingosine, thereby forming the ganglioside.

11. The method of making a ganglioside composition according to claim 10, the method further comprising:
(b) when the saccharyl moiety comprises a (Gal)ₙ-Gal moiety, wherein n is 1 or an integer greater than 1, contacting the ganglioside with a galactosidase, thereby cleaving the (Gal)ₙ moiety from the Gal moiety to which it is attached.

12. An improved method of preparing a ganglioside composition essentially free of terminal (Gal)ₙ, the improvement being, contacting a ganglioside with a terminal (Gal)ₙ with a galactosidase, thereby cleaving the (Gal)ₙ moiety and forming the ganglioside composition essentially free of terminal (Gal)ₙ.
